(19) United States
(12) Patent Application Publication
(10) Pub. No.: US 2026/0317429 A1
(43) Pub. Date: Oct. 15, 2026

(54) ANTI-FGFR2B Monoclonal Antibody

(71) Applicant: HUBEI BIO-PHARMACEUTICAL INDUSTRIAL TECHNOLOGICAL INSTITUTE INC., Wuhan, Hubei (CN)

(72) Inventors: Xuejun Zhang, Wuhan, Hubei (CN); Lifei Liu, Wuhan, Hubei (CN); Zhikui Cheng, Wuhan, Hubei (CN); Yong Zhao, Wuhan, Hubei (CN); Shifu Mo, Wuhan, Hubei (CN); Jun Yang, Wuhan, Hubei (CN); Lie Li, Wuhan, Hubei (CN); Kun He, Wuhan, Hubei (CN); Lingli Hu, Wuhan, Hubei (CN)

(73) Assignee: HUBEI BIO-PHARMACEUTICAL INDUSTRIAL TECHNOLOGICAL INSTITUTE INC., Wuhan, Hubei (CN)

(21) Appl. No.: 19/478,986

(22) PCT Filed: Apr. 26, 2024
(86) PCT No.: PCT/CN2024/090192

§ 371 Date: Oct. 27, 2025

(30) Foreign Application Priority Data
Apr. 28, 2023 (CN) 202310503469.6
Apr. 18, 2024 (CN) 202410479708.3

(51) Int. Cl. C07K 16/28; A61K 39/00; A61P 35/00; C07K 14/705; G01N 33/575

(52) U.S. Cl. CPC C07K 16/2863; A61P 35/00; C07K 14/7051; G01N 33/5758; A61K 2039/505; C07K 2317/24; C07K 2317/33; C07K 2317/565; C07K 2317/622; C07K 2317/732; C07K 2317/76; C07K 2317/92; G01N 2333/71

(57) ABSTRACT

Provided is an anti-FGFR2IIIb antibody. Specifically, the anti-FGFR2IIIb antibody can specifically bind to FGFR2IIIb, has high affinity and selectivity, and can efficiently block the binding of FGFR2IIIb to a ligand FGF1, FGF7 or FGF10 thereof. In addition, the antibody can also mediate, by means of an ADCC action, specific killing of FGFR2IIIb overexpressed cancer cells by immune cells. The antibody has excellent binding activity to FGFR2IIIb from multiple species origins. In vivo experiments show that the antibody has a significant anti-tumor effect on an FGFR2IIIb expressed gastric cancer-bearing mouse model, and has an application prospect in the field of tumor treatment.

### TECHNICAL FIELD

The invention relates to the field of biomedicine, in particular to an anti-FGFR2IIIb monoclonal antibody.

### BACKGROUND

Fibroblast Growth Factor Receptor (FGFR) belongs to a subfamily of tyrosine kinase receptor superfamily members. There are four FGFR genes (FGFR1-4) in the human genome.

FGFR-mediated signaling pathways are essential for normal cell growth and differentiation. They participate in physiological processes such as angiogenesis, cell proliferation and migration, regulation of organ development, and wound healing. However, when FGFRs are mutated or overexpressed, they can lead to overactivation of the FGFR signaling pathway, further inducing cancerous transformation of normal cells. Among these pathways, overactivation of RAS-RAF-MAPK stimulates cell proliferation and differentiation; overactivation of PI3K-AKT inhibits apoptosis; SATA is closely associated with promoting tumor invasion and metastasis and enhancing tumor immune evasion; and the PLCγ signaling pathway is a key pathway in regulating tumor cell metastasis.

FGFRs contain three extracellular immunoglobulin-like domains (D1-D3), a hydrophobic single transmembrane helix, and an intracellular tyrosine kinase domain, with an eight-residue acid box between D1 and D2. In FGFR1-3, ligand binding specificity is largely determined by alternative splicing at the C-terminus of the D3 domain. There are two alternative splices at the C-terminus of the D3 domain, encoded by exons 8 or 9, to generate either FGFRb or FGFRc isoforms. These b and c isoforms are typically restricted to epithelial and mesenchymal tissues, respectively. In this way, alternative splicing of the receptor allows ligands to activate the receptor in adjacent mesenchymal or epithelial tissues without initiating autocrine signaling.

The primary ligands for the FGFR2IIIb (or FGFR2b) form of FGFR2 are FGF1, FGF7, FGF10, and FGF22. FGFR2b can be overexpressed in tumors through FGFR2 gene amplification or transcriptional upregulation of the FGFR2b isoform, with FGFR2 amplification being the most common FGFR2 gene aberration. In the immunohistochemical section analysis of gastric cancer patients, FGFR2b expression can be detected in approximately 60% of tumor samples, among which 2%-9% of the samples show overexpression with the overexpression level ranging from 31% to 61%. These gastric cancers often exhibit a diffuse pattern and are associated with aggressive features of gastric cancer, including higher T stage, more frequent lymph node metastasis, and lower overall survival. In-depth research has confirmed that overexpression of the FGFR2b receptor or the amplification of the FGFR2 gene is directly associated with poor prognosis in gastric cancer patients. In addition to gastric cancer, abnormal activation of the FGF/FGFR2 signaling pathway has also been observed in other cancers, including but not limited to esophageal cancer, colorectal cancer, breast cancer, ovarian cancer, endometrial cancer, lung cancer (such as non-small cell lung cancer), cholangiocarcinoma, etc. Therefore, inhibiting FGFR2 signaling may be an effective mechanism of action for the treatment of various cancers.

### SUMMARY OF THE INVENTION

The purpose of the present invention is to provide an anti-FGFR2IIIb monoclonal antibody.

In the first aspect of the present invention, it provides an anti-FGFR2IIIb antibody comprising a heavy chain and a light chain, wherein the variable region of the heavy chain has complementarity determining regions (CDR) selected from the group consisting of:
(1) VH-CDR1 shown in SEQ ID NO: 6, VH-CDR2 shown in SEQ ID NO: 7, and VH-CDR3 shown in SEQ ID NO: 8, wherein the CDRs are defined according to the Kabat rule;
(2) VH-CDR1 shown in SEQ ID NO: 15, VH-CDR2 shown in SEQ ID NO: 16, and VH-CDR3 shown in SEQ ID NO: 8, wherein the CDRs are defined according to the Kabat rule;
(3) VH-CDR1 shown in SEQ ID NO: 6, VH-CDR2 shown in SEQ ID NO: 19, and VH-CDR3 shown in SEQ ID NO: 8, wherein the CDRs are defined according to the Kabat rule;
(4) VH-CDR1 shown in SEQ ID NO: 15, VH-CDR2 shown in SEQ ID NO: 19, and VH-CDR3 shown in SEQ ID NO: 8, wherein the CDRs are defined according to the Kabat rule;
(5) VH-CDR1 shown in SEQ ID NO: 15, VH-CDR2 shown in SEQ ID NO: 21, and VH-CDR3 shown in SEQ ID NO: 8, wherein the CDRs are defined according to the Kabat rule; and
(6) VH-CDR1 shown in SEQ ID NO: 9, VH-CDR2 shown in SEQ ID NO: 10, and VH-CDR3 shown in SEQ ID NO: 11, wherein the CDRs are defined according to the IMGT rule;
Furthermore, the variable region of the light chain has complementarity determining regions (CDR) selected from the group consisting of:
(1) VL-CDR1 shown in SEQ ID NO: 12, VL-CDR2 shown in SEQ ID NO: 13, and VL-CDR3 shown in SEQ ID NO: 14, wherein the CDRs are defined according to the Kabat rule;
(2) VL-CDR1 shown in SEQ ID NO: 17, VL-CDR2 shown in SEQ ID NO: 18, and VL-CDR3 shown in SEQ ID NO: 14, wherein the CDRs are defined according to the Kabat rule;
(3) VL-CDR1 shown in SEQ ID NO: 12, VL-CDR2 shown in SEQ ID NO: 18, and VL-CDR3 shown in SEQ ID NO: 20, wherein the CDRs are defined according to the Kabat rule; and
(4) VL-CDR1 shown in SEQ ID NO:40, VL-CDR2 represented by SAS, and VL-CDR3 shown in SEQ ID NO:14, wherein the CDRs are defined according to IMGT rule;
Furthermore, any one of the amino acid sequences in the above-mentioned CDR sequences also includes a derivative sequence that optionally has 1-2 amino acids added, deleted, modified, and/or substituted, and enables the derivative antibody composed of the heavy chain and light chain containing the derived CDR sequence to retain the binding affinity for FGFR2IIIb or its derivative protein.

In the first aspect of the present invention, it provides an anti-FGFR2IIIb antibody comprising a heavy chain and a light chain, wherein the variable region of the heavy chain has complementarity determining regions (CDR) selected from the group consisting of:
(1) VH-CDR1 shown in SEQ ID NO: 6, VH-CDR2 shown in SEQ ID NO: 7, and VH-CDR3 shown in SEQ ID NO: 8, wherein the CDRs are defined according to the Kabat rule;
(2) VH-CDR1 shown in SEQ ID NO: 15, VH-CDR2 shown in SEQ ID NO: 16, and VH-CDR3 shown in SEQ ID NO: 8, wherein the CDRs are defined according to the Kabat rule;
(3) VH-CDR1 shown in SEQ ID NO: 6, VH-CDR2 shown in SEQ ID NO: 19, and VH-CDR3 shown in SEQ ID NO: 8, wherein the CDRs are defined according to the Kabat rule;
(4) VH-CDR1 shown in SEQ ID NO: 15, VH-CDR2 shown in SEQ ID NO: 19, and VH-CDR3 shown in SEQ ID NO: 8, wherein the CDRs are defined according to the Kabat rule;
(5) VH-CDR1 shown in SEQ ID NO: 15, VH-CDR2 shown in SEQ ID NO: 21, and VH-CDR3 shown in SEQ ID NO: 8, wherein the CDRs are defined according to the Kabat rule; and
(6) VH-CDR1 shown in SEQ ID NO: 9, VH-CDR2 shown in SEQ ID NO: 10, and VH-CDR3 shown in SEQ ID NO: 11, wherein the CDRs are defined according to the IMGT rule;
Furthermore, the variable region of the light chain has complementarity determining regions (CDR) selected from the group consisting of:
(1) VL-CDR1 shown in SEQ ID NO: 12, VL-CDR2 shown in SEQ ID NO: 13, and VL-CDR3 shown in SEQ ID NO: 14, wherein the CDRs are defined according to the Kabat rule;
(2) VL-CDR1 shown in SEQ ID NO: 17, VL-CDR2 shown in SEQ ID NO: 18, and VL-CDR3 shown in SEQ ID NO: 14, wherein the CDRs are defined according to the Kabat rule;
(3) VL-CDR1 shown in SEQ ID NO: 12, VL-CDR2 shown in SEQ ID NO: 18, and VL-CDR3 shown in SEQ ID NO: 20, wherein the CDRs are defined according to the Kabat rule; and
(4) VL-CDR1 shown in SEQ ID NO:40, VL-CDR2 represented by SAS, and VL-CDR3 shown in SEQ ID NO:14, wherein the CDRs are defined according to IMGT rule.

In another preferred embodiment, the antibody has heavy chain variable regions CDRs (VH-CDR) and light chain variable regions CDRs (VL-CDR) selected from the group consisting of:
(1) VH-CDR1 shown in SEQ ID NO: 6, VH-CDR2 shown in SEQ ID NO: 7, and VH-CDR3 shown in SEQ ID NO: 8, VL-CDR1 shown in SEQ ID NO: 12, VL-CDR2 shown in SEQ ID NO: 13, and VL-CDR3 shown in SEQ ID NO: 14, wherein the CDRs are defined according to the Kabat rule;
(2) VH-CDR1 shown in SEQ ID NO: 15, VH-CDR2 shown in SEQ ID NO: 16, and VH-CDR3 shown in SEQ ID NO: 8, VL-CDR1 shown in SEQ ID NO: 17, VL-CDR2 shown in SEQ ID NO: 18, and VL-CDR3 shown in SEQ ID NO: 14, wherein the CDRs are defined according to the Kabat rule;
(3) VH-CDR1 shown in SEQ ID NO: 6, VH-CDR2 shown in SEQ ID NO: 19, and VH-CDR3 shown in SEQ ID NO: 8, VL-CDR1 shown in SEQ ID NO: 12, VL-CDR2 shown in SEQ ID NO: 18, and VL-CDR3 shown in SEQ ID NO: 20, wherein the CDRs are defined according to the Kabat rule;
(4) VH-CDR1 shown in SEQ ID NO: 15, VH-CDR2 shown in SEQ ID NO: 19, and VH-CDR3 shown in SEQ ID NO: 8, VL-CDR1 shown in SEQ ID NO: 12, VL-CDR2 shown in SEQ ID NO: 18, and VL-CDR3 shown in SEQ ID NO: 20, wherein the CDRs are defined according to the Kabat rule;
(5) VH-CDR1 shown in SEQ ID NO: 15, VH-CDR2 shown in SEQ ID NO: 21, and VH-CDR3 shown in SEQ ID NO: 8, VL-CDR1 shown in SEQ ID NO: 12, VL-CDR2 shown in SEQ ID NO: 18, and VL-CDR3 shown in SEQ ID NO: 20, wherein the CDRs are defined according to the Kabat rule;
(6) VH-CDR1 shown in SEQ ID NO: 15, VH-CDR2 shown in SEQ ID NO: 16, and VH-CDR3 shown in SEQ ID NO: 8, VL-CDR1 shown in SEQ ID NO: 12, VL-CDR2 shown in SEQ ID NO: 18, and VL-CDR3 shown in SEQ ID NO: 20, wherein the CDRs are defined according to the Kabat rule; and
(7) VH-CDR1 shown in SEQ ID NO: 9, VH-CDR2 shown in SEQ ID NO: 10, and VH-CDR3 shown in SEQ ID NO: 11, VL-CDR1 shown in SEQ ID NO:40, VL-CDR2 represented by SAS, and VL-CDR3 shown in SEQ ID NO:14, wherein the CDRs are defined according to the IMGT rule.

In another preferred embodiment, the antibody specifically binds to FGFR2IIIb or its derivative protein.

In another preferred embodiment, the antibody can specifically bind to FGFR2IIIb from human, mouse and cynomolgus monkey.

In another preferred embodiment, the FGFR2IIIb is FGFR2IIIb on the cell surface or soluble FGFR2IIIb.

In another preferred embodiment, the NCBI accession number of the FGFR2IIIb is NP_075259.

In another preferred embodiment, the FGFR2IIIb derivative protein is the FGFR2IIIb S252W mutant.

In another preferred embodiment, the affinity KD value of the antibody binding to FGFR2IIIb is ≤1×10⁻⁷ M, preferably ≤1×10⁻⁸ M, more preferably ≤5×10⁻⁹ M.

In another preferred embodiment, the antibody blocks the binding of FGFR2IIIb or its derivative protein with FGF1, FGF7 or FGF10.

In another preferred embodiment, the block refers to reducing the binding rate of the FGF1, FGF7 or FGF10 to FGFR2IIIb by 50%, preferably by 70%, and more preferably by 90%.

In another preferred example, the variable region of the heavy chain has the amino acid sequence of SEQ ID NO: 24, 26, 28, 30 or 31.

In another preferred embodiment, the heavy chain further comprises a heavy chain constant region.

In another preferred embodiment, the heavy chain constant region is of human or mouse origin.

In another preferred embodiment, the variable region of the light chain has the amino acid sequence of SEQ ID NO: 25, 27, or 29.

In another preferred embodiment, the light chain further comprises a light chain constant region.

In another preferred embodiment, the light chain constant region is of human or mouse origin.

In another preferred example, the heavy chain variable region of the antibody comprises the amino acid sequence of SEQ ID NO: 24, 26, 28, 30 or 31; and the light chain variable region of the antibody comprises the amino acid sequence of SEQ ID NO: 25, 27 or 29.

In another preferred example, the amino acid sequence of the heavy chain variable region has at least 80%, 85%, 90%, 91%, 92%, 93%, 94%, 95%, 96%, 97%, 98% or 99% sequence homology or sequence identity with the amino acid sequence shown in SEQ ID NO: 24, 26, 28, 30, 31 or 4 in the sequence listing.

In another preferred embodiment, the amino acid sequence of the light chain variable region has at least 80%, 85%, 90%, 91%, 92%, 93%, 94%, 95%, 96%, 97%, 98% or 99% sequence homology or sequence identity with the amino acid sequence shown in SEQ ID NO: 25, 27, 29 or 5 in the sequence listing.

In another preferred embodiment, the antibody has:
(1) the heavy chain variable region shown in SEQ ID NO: 24 and the light chain variable region shown in SEQ ID NO: 25;
(2) the heavy chain variable region shown in SEQ ID NO: 26 and the light chain variable region shown in SEQ ID NO: 27;
(3) the heavy chain variable region shown in SEQ ID NO: 28 and the light chain variable region shown in SEQ ID NO: 29;
(4) the heavy chain variable region shown in SEQ ID NO:30 and the light chain variable region shown in SEQ ID NO:29;
(5) the heavy chain variable region shown in SEQ ID NO:31 and the light chain variable region shown in SEQ ID NO:29;
(6) the heavy chain variable region shown in SEQ ID NO: 26 and the light chain variable region shown in SEQ ID NO: 29; or
(7) The heavy chain variable region shown in SEQ ID NO: 4 and the light chain variable region shown in SEQ ID NO: 5.

In another preferred embodiment, the heavy chain variable region of the antibody further comprises a human-derived framework region, and/or the light chain variable region of the antibody further comprises a human-derived framework region.

In another preferred embodiment, the heavy chain variable region of the antibody further comprises a mouse-derived framework region, and/or the light chain variable region of the antibody further comprises a mouse-derived framework region.

In another preferred embodiment, the antibody is selected from the group consisting of a mouse-derived antibody, a chimeric antibody, a humanized antibody, a fully human antibody, or a combination thereof.

In another preferred embodiment, the amino acid sequence of the heavy chain has at least 80%, 85%, 90%, 91%, 92%, 93%, 94%, 95%, 96%, 97%, 98% or 99% sequence homology or sequence identity with the amino acid sequence of SEQ ID NO: 32, 34, 36, 38, 39, or 22.

In another preferred embodiment, the amino acid sequence of the light chain has at least 80%, 85%, 90%, 91%, 92%, 93%, 94%, 95%, 96%, 97%, 98% or 99% sequence homology or sequence identity with the amino acid sequence of SEQ ID NO: 33, 35, 37 or 23.

In another preferred embodiment, the antibody has:
(1) a heavy chain shown in SEQ ID NO: 32 and a light chain shown in SEQ ID NO: 33;
(2) a heavy chain shown in SEQ ID NO:34 and a light chain shown in SEQ ID NO: 35;
(3) a heavy chain shown in SEQ ID NO: 36 and a light chain shown in SEQ ID 5 NO: 37;
(4) a heavy chain shown in SEQ ID NO: 38 and a light chain shown in SEQ ID NO: 37;
(5) a heavy chain shown in SEQ ID NO: 39 and a light chain shown in SEQ ID NO: 37;
(6) a heavy chain shown in SEQ ID NO: 34 and a light chain shown in SEQ ID NO: 37; or
(7) a heavy chain shown in SEQ ID NO: 22 and a light chain shown in SEQ ID NO: 23.

In the second aspect of the present invention, it provides a recombinant protein, wherein the recombinant protein comprises:
(i) the antibody of the first aspect of the present invention; and
(ii) optional tag sequence for assisting expression and/or purification.

In another preferred embodiment, the tag sequence comprises 6His tag, GGGS sequence, and FLAG tag.

In another preferred embodiment, the recombinant protein (or polypeptide) includes fusion protein.

In another preferred embodiment, the recombinant protein is a monomer, dimer, or polymer.

In another preferred embodiment, the recombinant protein is specific for anti-FGFR2IIIb.

In another preferred embodiment, the recombinant protein is a fusion protein.

In another preferred embodiment, the fusion protein is a monospecific antibody (ie, a monospecific antibody against FGFR2IIIb), a bispecific antibody, or a multispecific antibody (eg, a trispecific antibody).

In another preferred embodiment, the bispecific antibody or multispecific antibody not only targets FGFR2IIIb, but also specifically binds to additional target antigens (such as other tumor antigens, such as other antigens of gastric cancer or other tumors).

In the third aspect of the present invention, it provides a chimeric antigen receptor (CAR) construct, wherein the antigen binding region of the chimeric antigen receptor construct comprises a single-chain variable fragment (scFv) that specifically binds to FGFR2IIIb or its derivative protein, and the scFv has the heavy chain variable region and light chain variable region of the antibody of the first aspect of the present invention.

In the fourth aspect of the present invention, it provides a recombinant immune cell, which expresses an exogenous CAR construct of the third aspect of the present invention.

In another preferred embodiment, the immune cell is selected from the group consisting of NK cells, T cells.

In another preferred embodiment, the immune cells are derived from a human or non-human mammal, such as a mouse.

In the fifth aspect of the present invention, it provides a use of an active ingredient, wherein the active ingredient is selected from the group consisting of the antibody of the first aspect of the present invention, the recombinant protein of the second aspect of the present invention, the immune cell of the fourth aspect of the present invention, or a combination thereof, wherein the active ingredient is used for:
(a) preparing a diagnostic reagent, a detection plate or a kit; and/or
(b) preparing a drug for preventing and/or treating diseases associated with abnormal expression or function of FGFR2IIIb or its derivative protein.

In another preferred embodiment, the reagent is used to detect FGFR2IIIb or its derivative protein.

In another preferred embodiment, the reagent, detection plate or kit is used to detect diseases related to abnormal expression or function of FGFR2IIIb or its derivative protein.

In another preferred embodiment, the reagent, detection panel or kit is used to predict the risk of tumor or cancer.

In another preferred embodiment, the reagent is used for preventing and/or treating tumors or cancers.

In another preferred embodiment, the tumor includes solid tumors and blood cancers.

In another preferred embodiment, the tumor is a tumor that highly expresses FGFR2IIIb or its S252W mutant.

In another preferred embodiment, the tumor is selected from the group consisting of: gastric cancer, esophageal cancer, colorectal cancer, breast cancer, ovarian cancer, endometrial cancer, endometrial adenocarcinoma, cholangiocarcinoma, lung cancer, and non-small cell lung cancer.

In the sixth aspect of the present invention, it provides a pharmaceutical composition comprising:
(i) an active ingredient, which is selected from the group consisting of the antibody of the first aspect of the present invention, the recombinant protein of the second aspect of the present invention, the immune cell of the fourth aspect of the present invention, or a combination thereof; and
(ii) a pharmaceutically acceptable carrier.

In another preferred embodiment, the pharmaceutical composition is a liquid preparation.

In another preferred embodiment, the pharmaceutical composition is an injection.

In another preferred embodiment, the pharmaceutical composition comprises 0.01% to 99.99% of the antibody of the first aspect of the present invention, the recombinant protein of the second aspect of the present invention, the immune cell of the fourth aspect of the present invention, or a combination thereof, and 0.01% to 99.99% of a pharmaceutical carrier, where the percentages are the mass percentages relative to the total mass of the pharmaceutical composition.

In another preferred embodiment, the pharmaceutical composition is used for preventing and/or treating tumors or cancers.

In the seventh aspect of the present invention, it provides a polynucleotide encoding a polypeptide selected from the group consisting of:
(1) The antibody of the first aspect of the present invention;
(2) the recombinant protein of the second aspect of the present invention; or
(3) The chimeric antigen receptor (CAR) construct of the third aspect of the present invention.

In the eighth aspect of the present invention, it provides a vector comprising the polynucleotide of the seventh aspect of the present invention.

In another preferred embodiment, the vector includes: a bacterial plasmid, a bacteriophage, a yeast plasmid, a plant cell virus, a mammalian cell virus.

In another preferred embodiment, the vector is an adenovirus, a retrovirus, or other vectors.

In the ninth aspect of the present invention, it provides an engineered host cell, wherein the host cell comprising the vector of the eighth aspect of the present invention or has the polynucleotide of the seventh aspect of the present invention integrated into its genome.

In the tenth aspect of the present invention, it provides a method for detecting FGFR2IIIb or its derivative protein in a sample, wherein the method comprises the steps:
(1) contacting the sample with the antibody of the first aspect of the present invention;
(2) detecting the formation of an antigen-antibody complex, wherein the formation of the complex indicates the presence of FGFR2IIIb or its derivative protein in the sample.

In another preferred embodiment, the detection is for in vitro non-therapeutic and non-diagnostic purposes.

In the eleventh aspect of the present invention, it provides a composition for in vitro detection of FGFR2IIIb or its derivative protein in a sample, which comprises the antibody of the first aspect of the present invention, the recombinant protein of the second aspect of the present invention, the immune cell of the fourth aspect of the present invention, or a combination thereof as active ingredients.

In the twelfth aspect of the present invention, it provides a detection plate, which includes: a substrate (support plate) and a test strip, and the test strip contains the antibody of the first aspect of the present invention, the recombinant protein of the second aspect of the present invention, or a combination thereof.

In the thirteenth aspect of the present invention, it provides a kit, wherein the kit comprising:
(1) a first container containing the antibody of the first aspect of the present invention; and/or
(2) a second container containing a secondary antibody against the antibody of the first aspect of the present invention;
or,
the kit contains the detection plate of the twelfth aspect of the present invention.

In the fourteenth aspect of the present invention, it provides a method for preparing a recombinant polypeptide, wherein the method comprising:
(a) culturing the host cell of the ninth aspect of the present invention under conditions suitable for expression;
(b) isolating the recombinant polypeptide from the culture, wherein the recombinant polypeptide is the antibody of the first aspect of the present invention or the recombinant protein of the second aspect of the present invention.

In the fifteenth aspect of the present invention, it provides a use of the antibody of the first aspect of the present invention, or the recombinant protein of the second aspect of the present invention, or the immune cell of the fourth aspect of the present invention and/or the pharmaceutical composition of the sixth aspect of the present invention in the preparation of drugs for treating diseases related to abnormal expression or function of FGFR2IIIb.

In another preferred embodiment, the abnormal expression of FGFR2IIIb refers to overexpression of FGFR2IIIb.

In another preferred embodiment, the overexpression refers to a ratio of the expression level of FGFR2IIIb (F1) to the expression level under physiological conditions (F0) (ie, F1/F0) being ≥1.5, preferably ≥2, and more preferably ≥2.5.

In another preferred embodiment, the drug is used for preventing and/or treating tumors.

In another preferred embodiment, the drug is used for preventing and/or treat tumor occurrence, growth and/or metastasis.

In another preferred embodiment, the tumor includes solid tumors and blood cancers.

In another preferred embodiment, the tumor is a tumor that highly expresses FGFR2IIIb or its S252W mutant.

In another preferred embodiment, the tumor is selected from the group consisting of: gastric cancer, esophageal cancer, colorectal cancer, breast cancer, ovarian cancer, endometrial cancer, endometrial adenocarcinoma, cholangiocarcinoma, lung cancer, and non-small cell lung cancer.

In the sixteenth aspect of the present invention, it provides a method for treating a disease associated with abnormal expression or function of FGFR2IIIb or its derivative protein, which comprising administering an effective amount of the antibody of the first aspect of the present invention, or the recombinant protein of the second aspect of the present invention, or the immune cell of the fourth aspect of the present invention, or the pharmaceutical composition of the sixth aspect of the present invention, or a combination thereof to a subject in need thereof.

In another preferred embodiment, the disease associated with abnormal expression or function of FGFR2IIIb or its derivative protein is a tumor or cancer, preferably gastric cancer.

It should be understood that within the scope of the present invention, the various technical features of the present invention above and the various technical features specifically described hereinafter (as in the examples) may be combined with each other to constitute a new or preferred technical solution. And it needs not be described one by one, due to space limitations.

### DESCRIPTION OF THE DRAWINGS

The following drawings are used to illustrate specific embodiments of the present invention and are not used to limit the scope of the present invention defined by the claims.

FIG. 1 shows the binding activity of 27E9A11 to FGFR2IIIb-his.

FIG. 2 shows the inhibitory activity of 27E9A11 on FGF7/FGFR2IIIb binding.

FIG. 3 shows the inhibitory activity of 27E9A11 on FGF10/FGFR2IIIb binding.

FIG. 4 shows the binding activity of III-0 to human FGFR2IIIb protein.

FIG. 5 shows the activity of III-0 in blocking the binding of FGF1/FGFR2IIIb, the activity in blocking the binding of FGF7/FGFR2IIIb, and the activity in blocking the binding of FGF10/FGFR2IIIb.

FIG. 6 shows the binding activity of III-0 to SNU-16 cells.

FIG. 7 shows the binding activity of III-0 to CHO-FGFR2IIIb cells.

FIG. 8 shows the binding activities of III-0, III-10, III-11, III-14, and III-15 to human FGFR2IIIb and FGFR2IIIb (S252W) proteins.

FIG. 9 shows the binding of III-0, III-10, III-11, III-12, III-13, III-14, and III-15 to human FGFR2IIIc protein.

FIG. 10 shows the activity of III-0 and III-10 in blocking the binding of FGF1/FGFR2IIIb, blocking the binding of the FGF7/FGFR2IIIb, and blocking the binding of FGF10/FGFR2IIIb.

FIG. 11 shows the activities of III-11, III-12, III-13, III-14, and III-15 in blocking the binding of FGF1/FGFR2IIIb and the binding of FGF10/FGFR2IIIb.

FIG. 12 shows the binding activities of III-0, III-10, III-11, III-14, and III-15 to CHO-FGFR2IIIb.

FIG. 13 shows the binding activities of III-0, III-10, III-11, III-14, and III-15 to SNU-16.

FIG. 14 shows the ADCC activities of III-10, III-11, III-12, III-13, III-14, and III-15.

FIG. 15 shows the binding activity of III-10 to mouse and cynomolgus monkey FGFR2IIIb.

FIG. 16 shows the ability of III-10 to inhibit SNU16 tumor growth.

### MODES FOR CARRYING OUT THE INVENTION

Through extensive and deep research, the inventor accidentally obtains a class of monoclonal antibodies against FGFR2IIIb. The antibodies specifically bind to FGFR2IIIb with high affinity and are capable of effectively blocking the binding of FGFR2IIIb to its ligands (FGF1, FGF7, and FGF10). The antibodies of the present invention exhibit excellent binding activity against FGFR2IIIb from a variety of species. Furthermore, the antibodies of the present invention can mediate immune cell-specific killing of FGFR2IIIb-overexpressing cancer cells through ADCC. In vivo experiments have demonstrated significant tumor-suppressing effects in gastric cancer-bearing mouse models. The present invention has been completed on the basis of this.

### Term

In order to make the present invention easier to understand, certain technical and scientific terms are specifically defined below. Unless otherwise clearly defined in this article, all other technical and scientific terms used herein have the meanings generally understood by those of ordinary skill in the art to which the present invention belongs. Before describing the present invention, it should be understood that the present invention is not limited to the specific methods and experimental conditions described, because such methods and conditions can change. It should also be understood that the terms used herein are intended only to describe specific embodiments and are not intended to be restrictive, and the scope of the present invention will be limited only by the appended claims.

Unless otherwise defined, all technical and scientific terms used herein have the same meaning as commonly understood by one of ordinary skill in the art to which this invention belongs. As used herein, when used in reference to a specific recited value, the term “about” means that the value may vary by no more than 1% from the recited value. For example, as used herein, the expression “about 100” includes all values between 99 and 101 (e.g., 99.1, 99.2, 99.3, 99.4, etc.).

The amino acid three-letter code and one-letter code used in the present invention are as described in J. biol. chem, 243, p 3558 (1968).

As used herein, the term “treatment” refers to the administration of an internal or external therapeutic agent comprising the anti-FGFR2IIIb antibodies and a composition thereof of the present invention to a patient having one or more disease symptoms, in which the therapeutic agent is known to have a therapeutic effect. Generally, a therapeutic agent is administered to a patient in an amount (therapeutically effective amount) that effectively relieves one or more disease symptoms.

As used herein, the terms “optional” or “optionally” mean that the subsequently described event or circumstance may occur but not necessarily.

The “sequence identity” described in the present invention indicates the degree of identity between two nucleic acids or two amino acid sequences when optimally aligned and compared with appropriate mutations such as substitutions, additions or deletions. The sequence identity between the sequence described in the present invention and the sequences with identity to it can be at least 85%, 90%, or 95%, preferably at least 95%. Non-limiting examples include 85%, 86%, 87%, 88%, 89%, 90%, 91%, 92%, 93%, 94%, 95%, 96%, 97%, 98%, 99%, 100%.

### FGFR2IIIb

FGFR2IIIb is a transmembrane protein encoded by the FGFR2 gene (Fibroblast Growth Factor Receptor 2), with the NCBI accession number NP_075259. The protein consists of an extracellular domain, a hydrophobic single transmembrane helix, and a tyrosine kinase domain. The extracellular domain contains three relatively independent immunoglobulin-like domains (D1-D3). Ligand binds to the D2 domain, while the D3 domain regulates the binding activity of the receptor protein. The FGFR2 gene undergoes alternative splicing in different tissues, with the D3 domain existing in two different splice isoforms. Epithelial cells express D3 using exons 7 and 8, forming the FGFR2IIIb protein, while mesenchymal cells express D3 using exons 7 and 9, forming the FGFR2IIIc protein.

The FGFR2 gene harbors multiple pathogenic mutations in both somatic and cancer cells. Among them, the S252W mutation (serine to tryptophan at position 252) is the most common and well-studied mutation. The S252 site is located in exon 7, between the D2 and D3 domains. The S252 mutation alters ligand-receptor binding affinity, promoting ligand-independent FGFR2 dimerization and constitutive activation of the FGFR2 kinase, potentially leading to tumorigenesis and other pathologies. Endometrial adenocarcinoma is the most common tumor type harboring the FGFR2 S252W mutation. 12.52% of patients with this cancer harbor FGFR2 mutation, and the FGFR2 S252W mutation accounts for 4.71% of all endometrial adenocarcinomas. Apert syndrome (Apert syndrome), one of the most severe forms of human skull bone syndrome, is an autosomal dominant inherited disorder, with two-thirds of patients harboring the FGFR2 S252W mutation.

### Antibody

As used herein, the terms “antibody” or “immunoglobulin” are heterotetrameric glycoproteins of approximately 150,000 daltons with identical structural features, consisting of two identical light chains (L) and two identical heavy chains (H). Each light chain is linked to a heavy chain by one covalent disulfide bond, while the number of disulfide bonds varies between heavy chains of different immunoglobulin isotypes. Each heavy and light chain also has regularly spaced intrachain disulfide bonds. Each heavy chain has a variable region (VH) at one end, followed by multiple constant regions. Each light chain has a variable region (VL) at one end and a constant region at the other end; the constant region of the light chain is opposite the first constant region of the heavy chain, and the variable region of the light chain is opposite the variable region of the heavy chain. Specific amino acid residues form an interface between the variable regions of the light and heavy chains.

As used herein, the term “variable” refers to certain parts of the variable region in an antibody that are different in sequence, which form the binding and specificity of various specific antibodies to their specific antigens. However, variability is not evenly distributed throughout the variable region of an antibody. It is concentrated in three segments called complementarity determining regions (CDRs) or hypervariable regions in the light and heavy chain variable regions. The more conserved parts of the variable region are called framework regions (FRs). The variable regions of natural heavy and light chains each contain four FR regions, which are generally in a β-pleated configuration and are connected by three CDRs that form a connecting loop, and in some cases can form a partial β-pleated structure. The CDRs in each chain are closely together through the FR region and form the antigen-binding site of the antibody together with the CDRs of the other chain (see Kabat et al., NIH Publ. No. 91-3242, Volume 1, pages 647-669 (1991)). The constant regions do not directly participate in the binding of the antibody to the antigen, but they exhibit different effector functions, such as participating in the antibody-dependent cytotoxicity of the antibody.

The “light chains” of vertebrate antibodies (immunoglobulins) can be assigned to one of two distinct classes, called kappa and lambda, based on the amino acid sequence of their constant regions. Immunoglobulins can be divided into different classes based on the amino acid sequence of their heavy chain constant regions. There are five main classes of immunoglobulins: IgA, IgD, IgE, IgG, and IgM, some of which are further divided into subclasses (isotypes), such as IgG1, IgG2, IgG3, IgG4, IgA, and IgA2. The heavy chain constant regions corresponding to the different classes of immunoglobulins are called a, 8, &, Y, and u, respectively. The subunit structures and three-dimensional configurations of the different classes of immunoglobulins are well known in the art.

As used herein, the term “monoclonal antibody (mAb)” refers to an antibody obtained from a class of substantially homogeneous populations, i.e., the individual antibodies contained in the population are identical, except for a few possible naturally occurring mutations. Monoclonal antibodies are highly specific for a single antigenic site. Moreover, unlike ruleal polyclonal antibody preparations (which typically have different antibodies directed against different determinants), each monoclonal antibody is directed against a single determinant on the antigen. In addition to their specificity, the advantage of monoclonal antibodies is that they are synthesized by hybridoma culture and are not contaminated by other immunoglobulins. The modifier “monoclonal” indicates the characteristic of the antibody, being obtained from a substantially homogeneous population of antibodies, and this should not be construed as requiring any special method to produce the antibody.

Generally, antigen-binding properties of an antibody are described by three specific regions (known as the variable regions (CDRs)) located in the variable regions of the heavy and light chains. These regions are divided into four framework regions (FRs). The amino acid sequences of the four FRs are relatively conserved and do not directly participate in the binding reaction. These CDRs form a ring structure, spatially close to each other through the β-sheet formed by the FRs between them. The CDRs on the heavy chain and the corresponding CDRs on the light chain constitute the antigen-binding site of the antibody. The amino acid sequences of antibodies of the same type can be compared to determine which amino acids constitute the FR or CDR regions.

The term “antigen-binding fragment of an antibody” (or simply “antibody fragment”) refers to one or more fragments of an antibody that retain the ability to specifically bind to an antigen. It has been shown that fragments of full-length antibodies can be used to perform the antigen-binding function of an antibody. Examples of binding fragments encompassed by the term “antigen-binding fragment of an antibody” include (i) a Fab fragment, a monovalent fragment consisting of the VL, VH, CL, and CH1 domains; (ii) a F(ab′)2 fragment, a bivalent fragment comprising two Fab fragments linked by a disulfide bridge on the hinge region; (iii) a Fd fragment consisting of the VH and CH1 domains; and (iv) an Fv fragment consisting of the VH and VL domains of a single arm of an antibody. Fv antibodies are the smallest antibody fragments that contain the variable regions of the heavy and light chains of an antibody, but lack the constant region, and possess all the antigen-binding sites. Generally, Fv antibodies also contain a polypeptide linker between the VH and VL domains, and are capable of forming the structure required for antigen binding.

The present invention encompasses not only intact monoclonal antibodies, but also immunologically active antibody fragments or fusion proteins formed by antibodies with other sequences, such as Fab or (Fab′)2 fragments; antibody heavy chains; and antibody light chains. Therefore, the present invention also encompasses fragments, derivatives, and analogs of the antibodies.

The term “epitope” or “antigenic determinant” refers to a site on an antigen to which an immunoglobulin or antibody specifically binds. An epitope typically comprises at least 3, 4, 5, 6, 7, 8, 9, 10, 11, 12, 13, 14, or 15 contiguous or non-contiguous amino acids in a unique spatial conformation. An epitope can be a discontinuous three-dimensional site on an antigen that is recognized by the antibodies or antigen-binding fragments of the present invention.

The terms “specific binding”, “selective binding”, “selectively binds” and “specifically binds” refer to the binding of an antibody to a predetermined epitope on an antigen. Typically, antibodies bind with an affinity (KD) of about less than 10⁻⁷ M, such as about less than 10⁻⁸ M, 10⁻⁹ M, or 10⁻¹⁰ M or less.

In the present invention, antibodies include murine, chimeric, humanized or fully human antibodies prepared using techniques well known to those skilled in the art. Recombinant antibodies, such as chimeric and humanized monoclonal antibodies, including human and non-human parts, can be obtained by standard DNA recombinant techniques, and they are all useful antibodies. A chimeric antibody is a molecule in which different parts are derived from different animal species, such as a chimeric antibody having a variable region from a mouse monoclonal antibody and a constant region from a human immunoglobulin (see, for example, U.S. Pat. Nos. 4,816,567 and 4,816,397, which are hereby incorporated by reference in their entirety). A humanized antibody refers to an antibody molecule derived from a non-human species, having one or more complementary determining regions (CDRs) derived from a non-human species and a framework region derived from a human immunoglobulin molecule (see U.S. Pat. No. 5,585,089, which is hereby incorporated by reference in its entirety). These chimeric and humanized monoclonal antibodies can be prepared using DNA recombinant techniques well known in the art.

In the present invention, the antibodies can be monospecific, bispecific, trispecific, or more multispecific.

As used herein, the terms “heavy chain variable region” and “VH” are used interchangeably.

As used herein, the terms “variable region” and “complementarity determining region (CDR)” are used interchangeably.

The term “CDR” refers to the hypervariable region within the variable domain of an antibody that primarily contributes to antigen binding. One of the most commonly used definitions of CDRs is provided by Kabat E A et al. (1991) Sequences of proteins of immunological interest. NIH Publication 91-3242. Additionally, IMGT (Lefranc, 2003) and Chothia (Al-Lazikani, 1997) provide CDR definition rules, which are well known to those skilled in the art.

In a preferred embodiment of the present invention, the light chain of the antibody comprises the above-mentioned light chain variable region and light chain constant region, and the light chain constant region can be of mouse or human origin.

In the present invention, the antibody of the present invention further includes a conservative variant thereof, which means a polypeptide formed by replacing at most 10, preferably at most 8, more preferably at most 5, and most preferably at most 3 amino acids with amnio acid of similar properties as compared with the amino acid sequence of the antibody of the present invention. These conservative variant polypeptides are best produced by amino acid substitution according to Table A.

TABLE A

| | | Preferred |
|---|---|---|
| Initial residue | Representative substitution | substitution |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

As used herein, the term “ADCC” or “antibody-dependent cell-mediated cytotoxicity” includes cell-mediated reactions in which nonspecific cytotoxic cells expressing FcγRs recognize bound antibody on a target cell and cause lysis of the target cell. In various aspects, enhancing ADCC effector function can refer to enhanced potency or enhanced efficacy. “Potency” as used in an experimental context refers to the potency of an antibody when observing the efficacy of a specific therapeutic EC₅₀ “Efficacy” as used in the experimental context means the maximum possible effector function of an antibody at saturating levels.

### Anti-FGFR2IIIb Antibody

As used herein, the terms “antibody of the present invention”, “anti-FGFR2IIIb antibody of the present invention” and “FGFR2IIIb antibody of the present invention” are used interchangeably and all refer to the antibodies against FGFR2IIIb and its derivative protein (S252W mutant) described in the first aspect of the present invention.

The functions of the antibodies of the present invention are determined by the specific gene sequences of the light and heavy chain variable region genes. The antibodies of the present invention can specifically bind to FGFR2IIIb with high affinity and effectively block the binding of FGFR2IIIb to its ligands FGF1, FGF7, and FGF10. Using the variable region genes or complementarity-determining region (CDR) genes of the antibodies of the present invention, various genetically engineered antibodies can be engineered and produced in any expression system utilizing prokaryotic and eukaryotic cells.

The present invention provides an anti-FGFR2IIIb antibody, comprising a heavy chain and a light chain, wherein the variable region of the heavy chain has complementarity determining regions (CDR) selected from the group consisting of:
(1) VH-CDR1 shown in SEQ ID NO: 6, VH-CDR2 shown in SEQ ID NO: 7, and VH-CDR3 shown in SEQ ID NO: 8, wherein the CDRs are defined according to the Kabat rule;
(2) VH-CDR1 shown in SEQ ID NO: 15, VH-CDR2 shown in SEQ ID NO: 16, and VH-CDR3 shown in SEQ ID NO: 8, wherein the CDRs are defined according to the Kabat rule;
(3) VH-CDR1 shown in SEQ ID NO: 6, VH-CDR2 shown in SEQ ID NO: 19, and VH-CDR3 shown in SEQ ID NO: 8, wherein the CDRs are defined according to the Kabat rule;
(4) VH-CDR1 shown in SEQ ID NO: 15, VH-CDR2 shown in SEQ ID NO: 19, and VH-CDR3 shown in SEQ ID NO: 8, wherein the CDRs are defined according to the Kabat rule;
(5) VH-CDR1 shown in SEQ ID NO: 15, VH-CDR2 shown in SEQ ID NO: 21, and VH-CDR3 shown in SEQ ID NO: 8, wherein the CDRs are defined according to the Kabat rule; and
(6) VH-CDR1 shown in SEQ ID NO: 9, VH-CDR2 shown in SEQ ID NO: 10, and VH-CDR3 shown in SEQ ID NO: 11, wherein the CDRs are defined according to the IMGT rule;
Furthermore, the variable region of the light chain has complementarity determining regions (CDR) selected from the group consisting of:
(1) VL-CDR1 shown in SEQ ID NO: 12, VL-CDR2 shown in SEQ ID NO: 13, and VL-CDR3 shown in SEQ ID NO: 14, wherein the CDRs are defined according to the Kabat rule;
(2) VL-CDR1 shown in SEQ ID NO: 17, VL-CDR2 shown in SEQ ID NO: 18, and VL-CDR3 shown in SEQ ID NO: 14, wherein the CDRs are defined according to the Kabat rule;
(3) VL-CDR1 shown in SEQ ID NO: 12, VL-CDR2 shown in SEQ ID NO: 18, and VL-CDR3 shown in SEQ ID NO: 20, wherein the CDRs are defined according to the Kabat rule; and
(4) VL-CDR1 shown in SEQ ID NO: 40, VL-CDR2 represented by SAS, and VL-CDR3 shown in SEQ ID NO: 14, wherein the CDRs are defined according to IMGT rule.

Furthermore, the amino acid sequence also includes a sequence formed by adding, deleting, modifying and/or replacing at least one amino acid sequence, preferably an amino acid sequence with a homology or sequence identity of at least 80%, more preferably at least 85%, more preferably at least 90%, and most preferably at least 95%.

Methods for determining sequence homology or identity that are well known to those of ordinary skill in the art include, but are not limited to, Computational Molecular Biology, Lesk, A M, ed., Oxford University Press, New York, 1988; Biocomputing: Informatics and Genome Projects, Smith, D W, ed., Academic Press, New York, 1993; Computer Analysis of Sequence Data, Part I, Griffin, A M and Griffin, HG, eds., Humana Press, New Jersey, 1994; Sequence Analysis in Molecular Biology, von Heinje, G., Academic Press, 1987, and Sequence Analysis Primer, Gribskov, M. and Devereux, J., eds., Stockton Press, New York, 1991, and Carillo, H. and Lipman, D., SIAM J. Applied Biology. Math., 48:1073 (1988). The preferred method for determining identity is to obtain the largest match between the sequences tested. Methods for determining identity are compiled in publicly available computer programs. Preferred computer program methods for determining identity between two sequences include, but are not limited to, the GCG program package (Devereux, J. et al., 1984), BLASTP, BLASTN, and FASTA (Altschul, S, F. et al., 1990). The BLASTX program is publicly available from NCBI and other sources (BLAST Manual, Altschul, S. et al., NCBI NLM NIH Bethesda, Md. 20894; Altschul, S. et al., 1990). The well-known Smith-Waterman algorithm can also be used to determine identity.

Preferably, the antibody described herein is one or more of a full-length antibody protein, an antigen-antibody binding domain protein fragment, a bispecific antibody, a multispecific antibody, a single-chain antibody fragment (scFv), a single-domain antibody (sdAb), and a single-domain antibody (signal-domain antibody), as well as a monoclonal antibody or polyclonal antibody prepared from the above antibodies. The monoclonal antibody can be prepared by a variety of approaches and technologies, including hybridoma technology, phage display technology, single lymphocyte gene cloning technology, etc. The mainstream is to prepare monoclonal antibodies from wild-type or transgenic mice through hybridoma technology.

The full-length antibody protein is a conventional full-length antibody protein in the art, comprising a heavy chain variable region, a light chain variable region, a heavy chain constant region, and a light chain constant region. The heavy chain variable region and light chain variable region of the protein, together with the human heavy chain constant region and the human light chain constant region, constitute a fully human full-length antibody protein. Preferably, the full-length antibody protein is IgG1, IgG2, IgG3, or IgG4.

The antibody of the present invention may be a double-chain or single-chain antibody, and may be selected from animal-derived antibodies, chimeric antibodies, and humanized antibodies, more preferably humanized antibodies, human-animal chimeric antibodies, and even more preferably fully humanized antibodies.

The antibody derivatives of the present invention can be single-chain antibodies and/or antibody fragments, such as Fab, Fab′, (Fab′)2 or other antibody derivatives known in the art, as well as any one or more of IgA, IgD, IgE, IgG and IgM antibodies or other subtypes of antibodies.

The single-chain antibody is a conventional single-chain antibody in the art, which includes a heavy chain variable region, a light chain variable region and a short peptide of 15 to 20 amino acids.

The animal is preferably a mammal, such as a mouse.

The antibody of the present invention may be a chimeric antibody, a humanized antibody, a CDR-grafted and/or modified antibody targeting FGFR2IIIb (eg, human FGFR2IIIb, mouse FGFR2IIIb, or cynomolgus monkey FGFR2IIIb).

In the above content of the present invention, the number of added, deleted, modified and/or substituted amino acids is preferably not more than 40% (more preferably not more than 35%, more preferably 1-33%, more preferably 5-30%, more preferably 10-25%, more preferably 15-20%) of the total number of amino acids in the initial amino acid sequence.

In the above content of the present invention, more preferably, the number of amino acids added, deleted, modified and/or substituted may be 1-7, more preferably 1-5, more preferably 1-3, more preferably 1-2.

### Recombinant Protein

The present invention also provides a recombinant protein comprising one or more of the heavy chain CDR1 (VH-CDR1), heavy chain CDR2 (VH-CDR2) and heavy chain CDR3 (VH-CDR3) of the antibody of the present invention, and/or one or more of the light chain CDR1 (VL-CDR1), light chain CDR2 (VL-CDR2) and light chain CDR3 (VL-CDR3) of the antibody of the present invention.

Preferably, the recombinant protein further comprises an antibody heavy chain constant region and/or an antibody light chain constant region. The antibody heavy chain constant region is conventional in the art, preferably a rat-derived antibody heavy chain constant region or a human-derived antibody heavy chain constant region, more preferably a human-derived antibody heavy chain constant region. The antibody light chain constant region is conventional in the art, preferably a rat-derived antibody light chain constant region or a human-derived antibody light chain constant region, more preferably a human-derived antibody light chain constant region.

In another preferred embodiment, the recombinant protein comprises the antibody of the present invention.

The recombinant protein is a conventional protein in the art. Preferably, it is one or more of a full-length antibody protein, an antigen-antibody binding domain protein fragment, a bispecific antibody, a multispecific antibody, a single-chain antibody (scFv), a single-domain antibody (sdAb) and a single-domain antibody (Signle-domain antibody), as well as monoclonal antibodies or polyclonal antibodies prepared from the above antibodies.

The single-chain antibody is a conventional single-chain antibody in the art, which includes a heavy chain variable region, a light chain variable region and a short peptide of 15 to 20 amino acids.

The antigen-antibody binding domain protein fragment is a conventional antigen-antibody binding domain protein fragment in the art, which includes a light chain variable region, a light chain constant region, and an Fd segment of a heavy chain constant region. Preferably, the antigen-antibody binding domain protein fragment is Fab and F(ab′).

The single-domain antibody is a conventional single-domain antibody in the art, which includes a heavy chain variable region and a heavy chain constant region.

The single-domain antibody is a conventional single-domain antibody in the art, which only includes the heavy chain variable region.

The recombinant protein preparation method is a conventional preparation method in the art. The preparation method is preferably: isolating and obtaining from a transformant expressing the recombinant protein or obtaining by artificially synthesizing the protein sequence. The isolation and obtaining from a transformant expressing the recombinant protein are preferably performed by cloning a polynucleotide molecule encoding the protein and carrying a point mutation into a recombinant vector, transforming the resulting recombinant vector into a transformant to obtain a recombinant expression transformant, and culturing the resulting recombinant expression transformant to isolate and purify the recombinant protein.

### Polynucleotides

The present invention also provides a polynucleotide encoding the above-mentioned antibody or recombinant protein of the present invention or a chimeric antigen receptor (CAR) construct of the antibody of the present invention.

The method for preparing the polynucleotide is a conventional method in the art, and preferably includes the following steps: obtaining a nucleic acid molecule encoding the above protein by gene cloning technology, or obtaining a nucleic acid molecule encoding the above protein by artificial full sequence synthesis.

Those skilled in the art will appreciate that the base sequence encoding the amino acid sequence of the aforementioned protein can be appropriately substituted, deleted, altered, inserted, or added to provide a polynucleotide homolog. The polynucleotide homologs of the present invention can be prepared by substituting, deleting, or adding one or more bases in the gene encoding the protein sequence while maintaining antibody activity.

### Vector

The present invention also provides a recombinant expression vector comprising the polynucleotide.

The recombinant expression vector can be obtained by conventional methods in the art, i.e., by linking the polynucleotide molecules of the present invention to various expression vectors. The expression vector can be any conventional vector in the art, as long as it can carry the polynucleotide molecules. The vector preferably includes various plasmids, cosmids, phages, or viral vectors.

The present invention also provides a recombinant expression transformant comprising the above recombinant expression vector.

Wherein, the preparation method of the recombinant expression transformant is a conventional preparation method in the art, preferably: the above-mentioned recombinant expression vector is transformed into a host cell to obtain it. The host cell is various host cells conventional in the art, as long as it can satisfy the conditions for the above-mentioned recombinant expression vector to stably replicate on its own and the polynucleotide carried can be effectively expressed. Preferably, the host cell is E. coli TG1 or E. coli BL21 cell (expressing single-chain antibody or Fab antibody), or HEK293 or CHO cell (expressing full-length IgG antibody). The aforementioned recombinant expression plasmid is transformed into a host cell to obtain a preferred recombinant expression transformant of the present invention. The transformation method wherein is a conventional transformation method in the art, preferably a chemical transformation method, a heat shock method or an electroporation method.

### Antibody Preparation

The sequence of the DNA molecule of the antibody of the present invention or its fragment can be obtained by conventional techniques, such as PCR amplification or genomic library screening. In addition, the coding sequences of the light chain and heavy chain can be fused together to form a single-chain antibody.

Once the relevant sequence is obtained, it can be obtained in large quantities by recombinant methods. This is usually done by cloning it into a vector, then transferring it into cells, and then isolating the relevant sequence from the propagated host cells by conventional methods.

In addition, the sequences can also be synthesized by artificial synthesis, especially when the fragment length is shorter. Usually, a long fragment can be obtained by synthesizing multiple small fragments and then connecting them.

Currently, DNA sequences encoding the antibodies (or fragments thereof, or derivatives thereof) of the present invention can be obtained entirely through chemical synthesis. This DNA sequence can then be introduced into various existing DNA molecules (or vectors) and cells known in the art. In addition, mutations can also be introduced into the protein sequences of the present invention through chemical synthesis.

The present invention also relates to vectors comprising the above-mentioned appropriate DNA sequence and appropriate promoter or control sequence. These vectors can be used to transform appropriate host cells to enable them to express proteins.

The host cell can be a prokaryotic cell; or a lower eukaryotic cell; or a higher eukaryotic cell, such as a mammalian cell.

Typically, the transformed host cells are cultured under conditions suitable for expression of the antibodies of the present invention, and then purified to obtain the antibodies of the present invention using conventional separation and purification methods well known to those skilled in the art.

The resulting monoclonal antibodies can be characterized using conventional methods. For example, the binding specificity of the monoclonal antibodies can be determined using immunoprecipitation or in vitro binding assays such as radioimmunoassay (RIA) or enzyme-linked immunosorbent assay (ELISA). The binding affinity of the monoclonal antibodies can be determined, for example, using the Scatchard analysis of Munson et al., Anal. Biochem., 107:220 (1980).

The antibodies of the present invention can be expressed intracellularly, on the cell membrane, or secreted extracellularly. If necessary, the recombinant protein can be separated and purified by various separation methods utilizing its physical, chemical, and other properties. These methods are well known to those skilled in the art.

### Application

The present invention also provides uses of the antibodies, recombinant proteins, chimeric antigen receptor (CAR) constructs and/or immune cells of the present invention, for example, for preparing diagnostic preparations or preparing drugs.

Preferably, the drug is a drug for preventing and/or treating diseases associated with abnormal expression or function of FGFR2IIIb.

In the present invention, the disease associated with abnormal expression or function of FGFR2IIIb is a conventional disease associated with abnormal expression or function of FGFR2IIIb in the art. Preferably, the disease associated with abnormal expression or function of FGFR2IIIb is cancer.

In the present invention, the cancer is a conventional cancer in the art, preferably gastric cancer, esophageal cancer, colorectal cancer, breast cancer, ovarian cancer, endometrial cancer, endometrial adenocarcinoma, cholangiocarcinoma, lung cancer, and non-small cell lung cancer.

### Use for Detection and the Kit

The antibodies of the present invention can be used in detection applications, for example, for detecting a sample to provide diagnostic information.

In the present invention, the samples used include cells, tissue samples and biopsy specimens. The term “biopsy” used in the present invention should include all types of biopsies known to those skilled in the art. Therefore, the biopsy used in the present invention can include, for example, tumor resection samples, tissue samples prepared by endoscopic methods or organ puncture or needle biopsy.

Samples used in the present invention include fixed or preserved cell or tissue samples.

The present invention also provides a kit containing the antibody (or fragment thereof) of the present invention. In a preferred embodiment of the present invention, the kit further comprises a container, instructions for use, a buffer, etc. In a preferred embodiment, the antibody of the present invention can be fixed to a detection plate.

### Pharmaceutical Composition

The present invention also provides a composition. In a preferred embodiment, the composition is a pharmaceutical composition, which contains the above-mentioned antibody or its active fragment or fusion protein or corresponding immune cells, and a pharmaceutically acceptable carrier.

The antibodies of the present invention can also be expressed in cells by nucleotide sequences for cell therapy.

The pharmaceutical composition of the present invention is a pharmaceutical composition for preventing and/or treating diseases associated with abnormal expression or function of FGFR2IIIb or its derivative proteins.

The pharmaceutical compositions of the present invention contain a safe and effective amount of the monoclonal antibodies described above and a pharmaceutically acceptable carrier or excipient. The pharmaceutical formulation should be compatible with the route of administration. The active ingredient should be administered in a therapeutically effective amount. Furthermore, the polypeptides of the present invention may also be used in conjunction with other therapeutic agents.

In one embodiment of the present invention, the polypeptide of the present invention can be used in combination with other therapeutic agents for treating and/or preventing tumors.

In the present invention, preferably, the pharmaceutical composition of the present invention further comprises one or more pharmaceutical carriers. The pharmaceutical carriers are conventional pharmaceutical carriers in the art, and the pharmaceutical carriers can be any suitable physiologically or pharmaceutically acceptable pharmaceutical excipients. The pharmaceutical excipients are conventional pharmaceutical excipients in the art, and preferably include pharmaceutically acceptable excipients, fillers, or diluents.

In the present invention, the pharmaceutical composition is preferably administered in an effective amount, which is an amount capable of alleviating or delaying the progression of a disease, degenerative, or damaging condition. The effective amount can be determined on an individual basis and will be based in part on considerations of the symptoms to be treated and the desired outcome. Those skilled in the art can determine the effective amount by utilizing the above factors on an individual basis and using no more than routine experimentation.

The present invention provides use of the above-mentioned pharmaceutical composition in the preparation of a drug for preventing and/or treating a disease associated with abnormal expression or function of FGFR2 IIIb. Preferably, the disease associated with abnormal expression or function of FGFR2 IIIb is cancer. More preferably, the disease associated with abnormal expression or function of FGFR2 IIIb is gastric cancer.

### Method and Composition for Detecting FGFR2IIIb in Samples

The present invention also provides a method for detecting FGFR2IIIb in a sample (e.g., detecting cells overexpressing FGFR2IIIb), comprising the following steps: contacting the above-mentioned antibody with the sample to be tested in vitro, and detecting whether the above-mentioned antibody and the sample to be tested bind to form an antigen-antibody complex.

The meaning of overexpression is conventional in the art, and refers to the overexpression of FGFR2IIIb RNA or protein in the sample to be tested (due to increased transcription, post-transcriptional processing, translation, post-translational processing and changes in protein degradation), as well as local overexpression and increased functional activity (such as in the case of increased enzymatic hydrolysis of the substrate) due to changes in protein transport patterns (increased cell membrane localization).

In the present invention, the above-mentioned detection method for whether an antigen-antibody complex is formed by binding is a conventional detection method in the art, preferably a flow cytometry (FACS) detection.

The present invention provides a composition for detecting FGFR2IIIb in a sample, comprising the above-mentioned antibody, recombinant protein, immune cell, or a combination thereof as an active ingredient. Preferably, it also comprises a compound composed of a functional fragment of the above-mentioned antibody as an active ingredient.

### The Main Advantages of the Present Invention Include:

1) The anti-FGFR2IIIb antibody of the present invention can bind to FGFR2IIIb with high affinity and can effectively block the binding of FGFR2IIIb to its ligands (FGF1, FGF7 and FGF10).

2) The antibodies of the present invention have excellent binding activity to FGFR2IIIb from multiple species and excellent specificity, and essentially do not bind to FGFR2IIIc; the antibodies of the present invention have good selectivity for FGFR2IIIb relative to FGFR2IIIc.

3) The antibodies of the present invention can mediate immune cell-specific killing of FGFR2IIIb-overexpressing cancer cells through ADCC.

4) The antibody of the present invention has a significant tumor-suppressing effect in a tumor-bearing mouse model, and the antibody of the present invention has a significant tumor-suppressing effect even at a low dose.

The invention is further illustrated below in conjunction with specific embodiments. The present disclosure is further set forth below with reference to specific embodiments. The conditions of the experimental methods not specifically indicated in the following examples are usually in accordance with conventional conditions as described in Sambrook et al., Molecular Cloning: A Laboratory Manual (New York: Cold Spring Harbor Laboratory Press, 1989), or according to the conditions recommended by the manufacturer. Percentages and parts are by weight unless otherwise stated.

### Example 1: Acquisition of Anti-Human FGFR2IIIb Antibodies

The present inventors immunized 6-8 weeks old female Balb/c mice with FGFR2IIIb (D2+D3) (SEQ ID NO: 1) as an antigen. Splenocytes from the immunized mice were fused with SP2/0-AG14 cells for hybridoma cell fusion, and an appropriate amount of the fused cells were plated onto 96-well plates. 7 to 10 days after fusion, the supernatant from each well was collected and the hybridoma cells were assayed by ELISA for binding activity of mouse antibodies secreted by the hybridoma cells to human FGFR2IIIb-his (ECD) (SEQ ID NO: 2) and FGFR2IIIc-his (ECD) (SEQ ID NO: 3), as well as for their inhibitory activity against FGF7/FGFR2IIIb and FGF10/FGFR2IIIb binding (FGF7 and FGF10 were purchased from Nearshore Protein, Cat. No. CM88 and CR11, respectively). Multiple hybridoma cells were ultimately obtained that specifically bound to FGFR2IIIb-his (ECD) but not to FGFR2IIIc-his (ECD). The hybridoma cell with the best inhibitory activity for FGF7/FGFR2IIIb and FGF10/FGFR2IIIb binding was 27E9A11. Sequencing revealed an antibody secreted by this hybridoma cell, designated 27E9A11. The cDNA sequences of the heavy and light chain variable region cDNA sequences corresponding to the 27E9A11 antibody encode the heavy chain variable region sequence of SEQ ID NO:4, and the light chain variable region cDNA sequence of SEQ ID NO:5. The complementary region determining regions (CDRs) of the 27E9A11 antibody are shown in Table 1.

TABLE 1
Antigenic complementarity determinants (CDRs)
of mouse antibody 27E9A11

| Coding | | | |
|---|---|---|---|
| rules | VHCDR1 | VHCDR2 | VHCDR3 |
| Kabat | NYNMH | AIYPGNGDT | GDFGY |
| | (SEQ ID | SYNQKFKG | (SEQ ID |
| | NO: 6) | (SEQ ID NO: 7) | NO: 8) |
| IMGT | GYTFANYN | IYPGNGDT | ARGDFGY |
| | (SEQ ID | (SEQ ID | (SEQ ID |
| | NO: 9) | NO: 10) | NO: 11) |
| | VLCDR1 | VLCDR2 | VLCDR3 |
| Kabat | KASQDVNIDVA | SASYRYT | QQHYSTPYT |
| | (SEQ ID | (SEQ ID | (SEQ ID |
| | NO: 12) | NO: 13) | NO: 14) |
| IMGT | QDVNID | SAS | QQHYSTPYT |
| | (SEQ ID | (SEQ ID | (SEQ ID |
| | NO: 40) | | NO: 14) |

### Detection of the Binding Activity of 27E9A11 Antibody to Human FGFR2IIIb Protein

A 96-well high-affinity plate was coated with 100 μL/well of a 1 μg/mL human FGFR2 IIIb-his protein solution and shaken overnight at 4° C. The next day, the plate was washed three times with 300 μL of PBST (Tween 20:0.5% 0) and then blocked with 100 μL/well of 5% BSA/PBS for 2 hours at room temperature. The plate was washed three times with 300 μL of PBST. A serial dilution of the 27E9A11 antibody sample was prepared in PBS. 100 μL/well of this solution was added to the 96-well plate and shaken for 1 hour at room temperature. The plate was washed three times with 300 μL of PBST. A secondary antibody, goat anti-mouse IgG HRP solution (Thermo Fisher, Cat. No. A16090, similarly below), was prepared and added to the 96-well plate at 100 μL/well. The plate was shaken for 30 minutes at room temperature. The plate was washed four times with 300 μL of PBST. Add 100 μL/well TMB (tetramethylbenzidine) and color for 3 minutes. Add 100 μL/well 0.6N H₂SO₄ to stop color development and measure OD_{450 nm}.

### Detection of the 27E9A11 Antibody Blocking the Binding of Human FGF Factors (FGF7 or FGF10) to Human FGFR2IIIb Protein

Coat a 96-well high-affinity plate with 100 μL/well of 1 μg/mL human FGF and 5 μg/mL heparin sodium solution (Sinopharm, Cat. No. 63007131, same below), and then incubate at 4° C. with shaking overnight. The next day, wash the plate three times with 300 μL of PBST (Tween 20:0.5% %) and then block with 100 μL/well of 5% BSA/PBS for 2 hours at room temperature with shaking. Wash the plate three times with 300 μL of PBST. Prepare a serial dilution of the 27E9A11 antibody sample in PBS and add 1 μg/mL FGFR2IIIb-huFc in a 1:1 ratio, premixing thoroughly. Add 100 μL/well of the mixture to the 96-well plate and shake at room temperature for 2 hours. Wash the plate three times with 300 μL of PBST. Prepare a secondary antibody, goat anti-human IgG HRP solution (Abcam, cat. no. ab6858, same below). Add 100 μL/well of the solution to a 96-well plate, and shake at room temperature for 1 hour. Wash the plate four times with 300 μL PBST. Add 100 μL/well TMB (tetramethylbenzidine) and develop for 5 minutes. Add 100 μL/well 0.6N H₂SO₄ to stop the color development and measure the OD_{450 nm}.

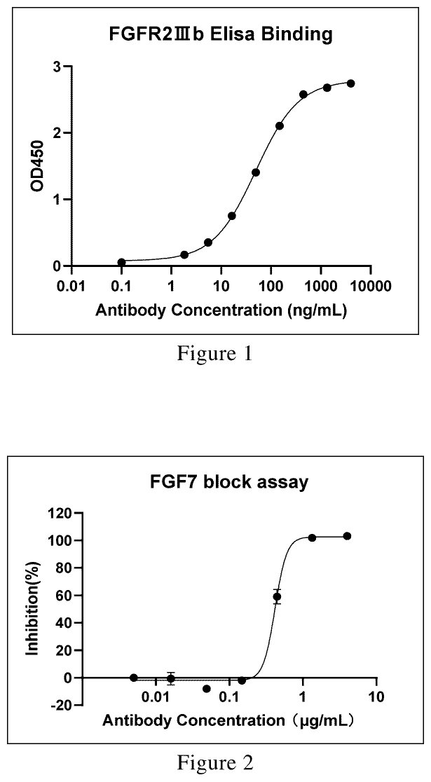

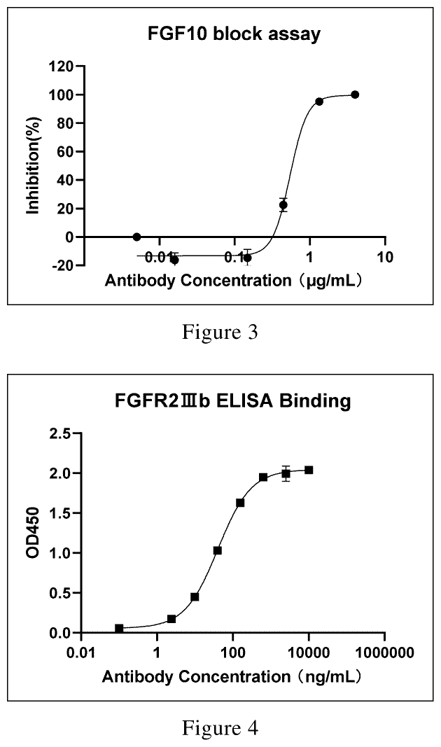

Results: The binding activity of 27E9A11 antibody to FGFR2IIIb-his is shown in FIG. 1, and the EC₅₀ is shown in Table 2. The inhibitory activity of 27E9A11 antibody on FGF7/FGFR2IIIb binding is shown in FIG. 2, and IC₅₀ is shown in Table 2. The inhibitory activity of 27E9A11 antibody on FGF10/FGFR2IIIb binding is shown in FIG. 3, and IC₅₀ is shown in Table 2.

TABLE 2
Binding and blocking abilities of antibody 27E9A11

| | | IC₅₀ | IC₅₀ |
|---|---|---|---|
| Antibody | EC₅₀ (FGFR2b) | (FGF7/FGFR2b) | (FGF10/FGFR2b) |
| 27E9A11 | 0.050 μg/mL | 0.417 μg/mL | 0.545 μg/mL |

### Example 2: Binding of Chimeric Antibody III-0 to Human FGFR2IIIb Protein

The heavy chain variable region and light chain variable region of mouse antibody 27E9A11 were linked to the constant region of the human IgG1 heavy chain and the constant region of the human K chain, respectively, to obtain human-mouse chimeric antibody III-0 with the heavy chain sequence of SEQ ID NO:22 and the light chain sequence of SEQ ID NO:23.

The binding activity of III-0 to human FGFR2IIIb protein was studied. The detection method is shown in Example 1 (the secondary antibody was changed to goat anti-human IgG HRP). The results are shown in FIG. 4. III-0 can effectively bind to human FGFR2IIIb protein with an EC₅₀ of 40.42 ng/ml.

### Example 3: III-0 Blocks the Binding of Human FGF Factors (FGF1/FGF7/FGF10) to Human FGFR2IIIb Protein

Coat a 96-well high-affinity plate with 100 μL/well of a solution containing 1 μg/mL human FGF and 5 μg/mL sodium heparin, and then incubate at 4° C. overnight with shaking. The next day, wash three times with 300 μL of PBST (Tween 20:0.5% 0) and then block with 100 μL/well of 5% BSA/PBS for 2 hours at room temperature with shaking. Wash the plate three times with 300 μL of PBST. Prepare a serial dilution of the antibody sample in PBS and add 1 μg/mL FGFR2IIIb-mFc at a 1:1 ratio. Add 100 μL/well of the mixture to the 96-well plate and shake at room temperature for 2 hours. Wash the plate three times with 300 μL of PBST. Prepare a secondary antibody, goat anti-mouse IgG HRP solution, and add 100 μL/well of the solution to the 96-well plate and shake at room temperature for 1 hour. Wash the plate four times with 300 μL of PBST. Add 100 μL/well TMB (tetramethylbenzidine) and color for 5 minutes. Add 100 μL/well 0.6N H₂SO₄ to stop color development and measure OD_{450 nm}.

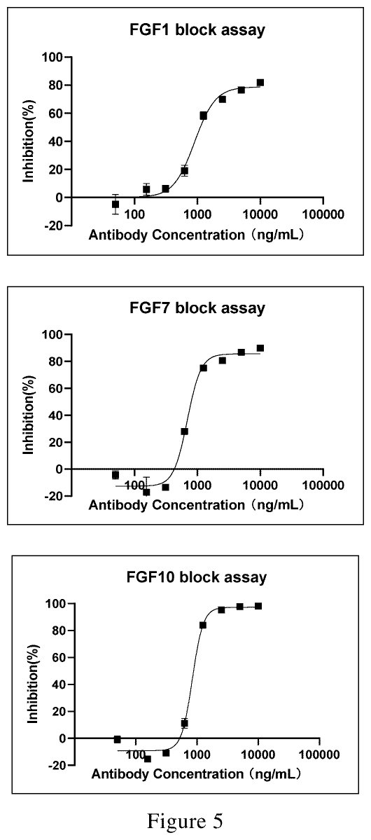

The results are shown in FIG. 5, the III-0 can effectively block the binding of FGF1/7/10 to FGFR2IIIb. Its IC₅₀ is shown in Table 3.

TABLE 3
Blocking ability of antibody III-0

| | IC₅₀ | IC₅₀ | IC₅₀ |
|---|---|---|---|
| Antibody | (FGF1/FGFR2b) | (FGF7/FGFR2b) | (FGF10/FGFR2b) |
| III-0 | 0.904 μg/mL | 0.694 μg/mL | 0.840 μg/mL |

### Example 4: Binding of III-0 to FGFR2IIIb Protein on SNU-16 Cells

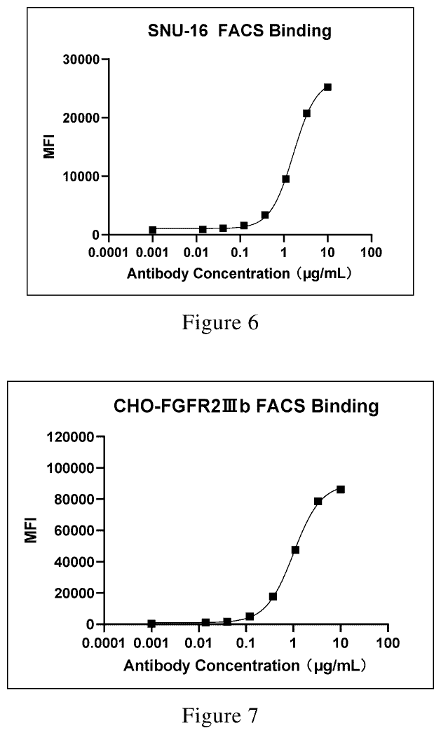

SNU-16 cells are derived from human gastric cancer and naturally overexpress human FGFR2IIIb protein. SNU-16 cells were digested and centrifuged, and resuspend in PBS to a density of 2×10⁶ cells/mL. Mix it well, and transfer 100 μL of the cell suspension to a centrifuge tube. Centrifuge and discard the supernatant. Prepare a gradient dilution solution of the antibody sample in PBS. Add 100 μL of each diluted solution to each centrifuge tube and shake at 4° C. for 1 hour. Centrifuge the cells, discard the supernatant, pipet and mix with 400 μL PBS, and repeat these steps 3 times. Prepare the secondary goat anti-human IgG (H+L) flow cytometry detection antibody (Thermo Fisher, cat. no. A21091, the same below), add 100 μL of this secondary antibody solution to each centrifuge tube, and shake it at 4° C. for 30 minutes. Centrifuge the cells at 2000 rpm for 3 minutes, discard the supernatant, pipet and mix with 400 μL PBS, and repeat these steps 2 times. Transfer the cell suspensions to flow cytometry (Beckman, Cytoflex) for detection. The results are shown in FIG. 6. III-0 can effectively recognize and bind to SNU-16 cells, and the bound EC₅₀ is shown in Table 4.

### Example 5: Binding of Chimeric Antibodies to FGFR2IIIb Protein on CHO-FGFR2IIIb Cells

The inventors constructed a CHO-K1 cell line that overexpressed FGFR2IIIb protein. CHO-FGFR2IIIb cells were digested and centrifuged, and resuspend in PBS to a density of 2×10⁶ cells/mL. Mix it well, and transfer 100 μL of the cell suspension to a centrifuge tube. Centrifuge and discard the supernatant. Prepare a gradient dilution solution of the antibody sample in PBS. Add 100 μL of each diluted solution to each centrifuge tube and shake at 4° C. for 1 hour. Centrifuge the cells, discard the supernatant, mix well with 400 μL PBS, and repeat these steps 3 times. Prepare the secondary goat anti-human IgG (H+L) flow cytometry detection antibody, add 100 μL of this secondary antibody solution to each centrifuge tube, and shake it at 4° C. for 30 minutes. Centrifuge the cells at 2000 rpm for 3 minutes, discard the supernatant, mix well with 400 μL PBS, and repeat these steps 2 times. Transfer the cell suspensions to flow cytometry (Beckman, Cytoflex) for detection. The results are shown in FIG. 7. III-0 can effectively recognize and bind to CHO-FGFR2IIIb cells, and the bound EC₅₀ is shown in Table 4.

TABLE 4
Binding ability of antibody III-0 to different cells

| Antibody | EC₅₀ (SNU-16) | EC₅₀ (CHO-FGFR2b) |
|---|---|---|
| III-0 | 1.641 μg/mL | 1.010 μg/mL |

### Example 6: Binding of Humanized Antibodies to Human FGFR2IIIb and FGFR2IIIb (S252W) Proteins

The chimeric antibody III-0 was humanized using CDR grafting to generate the humanized antibody III-10. The III-10 then underwent affinity maturation. Specifically, single-point saturation mutagenesis was performed at each amino acid position in the CDR region of the III-10 antibody. Mutation hotspots with antigen-specific binding ability were screened using ELISA, and these hotspots were then combined to generate candidate antibody mutation sequences. The affinity of the candidate antibodies for the antigen was tested using SPR. Five antibodies were finally obtained, designated as III-11, III-12, III-13, III-14, and III-15. The sequences of these antibodies are listed in the table below.

TABLE 5
Affinity-engineered antibody variable region sequences
(CDR sequences defined by Kabat rules)

| | | | CDR3 | Variable | Full |
|---|---|---|---|---|---|
| | CDR1 | CDR2 | (SEQ ID | region | length |
| Clone | (SEQ ID | (SEQ ID | NO) | (SEQ ID | (SEQ ID |
| name | NO) | NO) | | NO) | NO) |
| Heavy | NYNMH | AIYPGNGDTS | GDFGY | 24 | 32 |
| chain | (6) | YNQKFKG | (8) | | |
| of III-10 | | (7) | | | |
| Light | KASQDVNI | SASYRYT | QQHYSTP | 25 | 33 |
| chain | DVA | (13) | YT | | |
| of III-10 | (12) | | (14) | | |
| Heavy | IYNMH | AIYPDNGDTF | GDFGY | 26 | 34 |
| chain | (15) | YNQKFKG | (8) | | |
| of III-11 | | (16) | | | |
| Light | KASQDVNI | LASYRYT | QQHYSTP | 27 | 35 |
| chain | DPA | (18) | YT | | |
| of III-11 | (17) | | (14) | | |
| Heavy | NYNMH | AIYPDNGDTS | GDFGY | 28 | 36 |
| chain | (6) | YNQKFKG | (8) | | |
| of III-12 | | (19) | | | |
| Light | KASQDVNI | LASYRYT | QQTYSTP | 29 | 37 |
| chain | DVA | (18) | YT | | |
| of III-12 | (12) | | (20) | | |
| Heavy | IYNMH | AIYPDNGDTS | GDFGY | 30 | 38 |
| chain | (15) | YNQKFKG | (8) | | |
| of III-13 | | (19) | | | |
| Light | KASQDVNI | LASYRYT | QQTYSTP | 29 | 37 |
| chain | DVA | (18) | YT | | |
| of III-13 | (12) | | (20) | | |
| Heavy | IYNMH | AIYPGNGDTF | GDFGY | 31 | 39 |
| chain | (15) | YNQKFKG | (8) | | |
| of III-14 | | (21) | | | |
| Light | KASQDVNI | LASYRYT | QQTYSTP | 29 | 37 |
| chain | DVA | (18) | YT | | |
| of III-14 | (12) | | (20) | | |
| Heavy | IYNMH | AIYPDNGDTF | GDFGY | 26 | 34 |
| chain | (15) | YNQKFKG | (8) | | |
| of III-15 | | (16) | | | |
| Light | KASQDVNI | LASYRYT | QQTYSTP | 29 | 37 |
| chain | DVA | (18) | YT | | |
| of III-15 | (12) | | (20) | | |

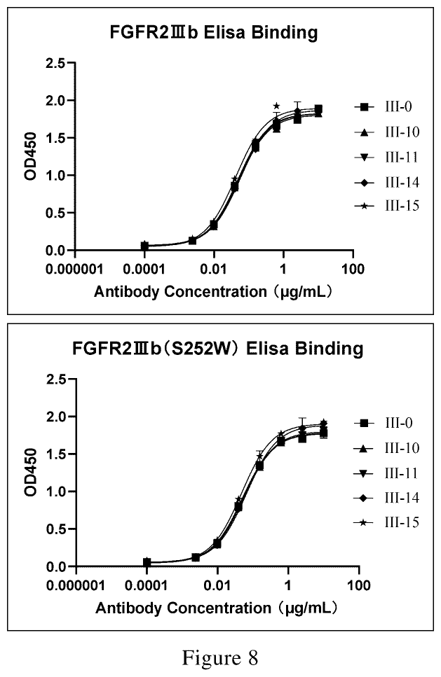

S252W is a common mutation in FGFR2IIIb on tumors. The binding activity of a series of humanized antibodies of III-0 to FGFR2IIIb and FGFR2IIIb (S252W) was tested. The detection method is shown in Example 2. The test results are shown in FIG. 8. The binding activity of the humanized antibody of III-0 to human FGFR2IIIb and FGFR2IIIb (S252W) protein is comparable to that of III-0. The EC₅₀ values of each antibody is shown in Table 6.

TABLE 6
bingding ability of affinity-modified antibodies to
FGFR2IIIb/FGFR2IIIb(S252W)

| | EC₅₀ (μg/mL) | | | | |
|---|---|---|---|---|---|
| Protein | III-0 | III-10 | III-11 | III-14 | III-15 |
| FGFR2IIIb | 0.048 | 0.052 | 0.051 | 0.058 | 0.042 |
| FGFR2IIIb(S252W) | 0.050 | 0.055 | 0.056 | 0.066 | 0.048 |

### Example 7: Binding of Anti-Human FGFR2 IIIb Antibody to Human FGFR2 IIIc Protein

Coat a 96-well high-affinity plate with 100 μL/well of a 1 μg/mL human FGFR2IIIc protein solution, and then incubate at 4° C. with shaking overnight. The next day, wash the plate three times with 300 μL PBST (Tween20: 0.5% %), then block it with 100 μL/well of 5% BSA/PBS for 2 hours and shake it at room temperature. Wash the plate three times with 300 μL PBST. Prepare a gradient dilution solution of the antibody sample with PBS. Add the solution to a 96-well plate at 100 μL/well and shake at room temperature for 1 hour. Wash the plate three times with 300 μL PBST. Prepare a secondary antibody goat anti-human IgG HRP solution and add it to a 96-well plate at 100 μL/well and shake it at room temperature for 30 minutes. Wash the plate four times with 300 μL PBST. Add 100 μL/well TMB (tetramethylbenzidine) and develop color for 3 minutes. Add 100 μL/well 0.6N H₂SO₄ to stop color development and detect OD_{450 nm}.

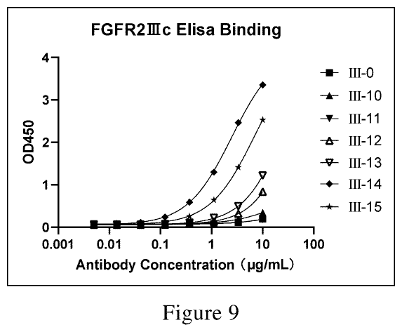

As shown in FIG. 9, the test results show that III-0, III-10, and III-11 showed weak or no binding to human FGFR2 IIIc protein. In contrast, III-12, III-13, III-14, and III-15 showed relatively weak binding activity to human FGFR2 IIIc protein. These results demonstrate that the antibodies of the present invention have high specificity and can reduce unwanted binding, mitigate potential side effects, and improve drug safety in clinical applications.

### Example 8: Humanized Antibodies Block the Binding of Human FGF Factors (FGF1/FGF7/FGF10) to Human FGFR2IIIb Protein

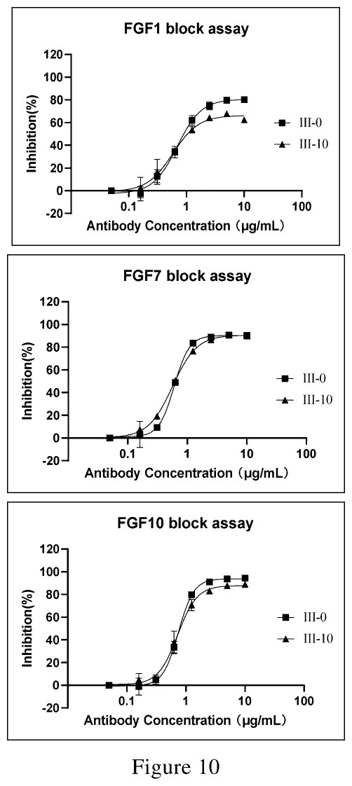

The specific detection method is shown in Example 3. The detection results are shown in FIG. 10. Both III-10 and III-0 can block the binding of FGF1/FGFR2IIIb. The activity of III-10 in blocking the binding of FGF10/FGFR2IIIb is equivalent to that of III-0. IC₅₀ is shown in Table 7.

TABLE 7
Blocking ability of antibodies III-0 and III-10

| | IC₅₀ | IC₅₀ | IC₅₀ |
|---|---|---|---|
| Antibody | (FGF1/FGFR2b) | (FGF7/FGFR2b) | (FGF10/FGFR2b) |
| III-0 | 0.692 μg/mL | 0.603 μg/mL | 0.745 μg/mL |
| III-10 | 0.581 μg/mL | 0.565 μg/mL | 0.724 μg/mL |

### Example 9: Humanized Antibodies (Affinity Maturation) Block the Binding of Human FGF Factors (FGF1/FGF10) to Human FGFR2IIIb Protein

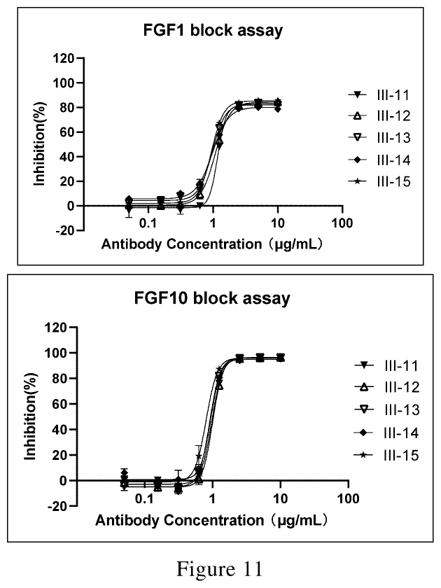

The specific detection method is shown in Example 3. The detection results are shown in FIG. 11. III-11, III-12, III-13, III-14, and III-15 can all block the binding of FGF1/10 to FGFR2IIIb, and the blocking ability is equivalent. Details can be found in Table 8.

TABLE 8
Blocking ability of affinity matured antibodies

| Antibody | IC₅₀ (FGF1/FGFR2b) | IC₅₀ (FGF10/FGFR2b) |
|---|---|---|
| III-11 | 1.179 μg/mL | 0.976 μg/mL |
| III-12 | 1.083 μg/mL | 0.999 μg/mL |
| III-13 | 0.966 μg/mL | 0.905 μg/mL |
| III-14 | 0.974 μg/mL | 0.937 μg/mL |
| III-15 | 0.941 μg/mL | 0.805 μg/mL |

### Example 10: Binding of Humanized Antibodies to FGFR2IIIb Protein on CHO-FGFR2IIIb Cells

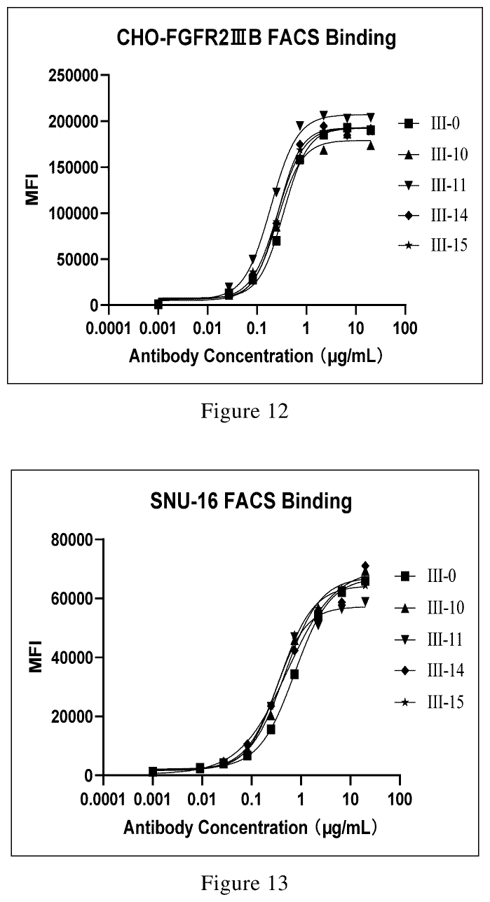

The specific detection method is shown in Example 5. The results are shown in FIG. 12. The binding activity of III-10, III-11, III-14, and III-15 to CHO-FGFR2IIIb are equivalent to that of III-0. The EC₅₀ of each antibody is shown in Table 9.

### Example 11: Binding of Humanized Antibodies to FGFR2IIIb Protein on SNU-16 Cells

The specific detection method is shown in Example 4. The results are shown in FIG. 13. The binding activity of III-10, III-11, III-14, and III-15 in binding to SNU-16 are equivalent to that of III-0. The EC₅₀ of each antibody is shown in Table 9.

TABLE 9
Binding ability of humanized antibodies to different cells

| Antiody | EC₅₀ (CHO-FGFR2b) | EC₅₀ (SNU-16) |
|---|---|---|
| III-0 | 0.342 μg/mL | 0.740 μg/mL |
| III-10 | 0.257 μg/mL | 0.471 μg/mL |
| III-11 | 0.190 μg/mL | 0.319 μg/mL |
| III-14 | 0.263 μg/mL | 0.526 μg/mL |
| III-15 | 0.260 μg/mL | 0.378 μg/mL |

### Example 12: ADCC Effect of Humanized Antibodies

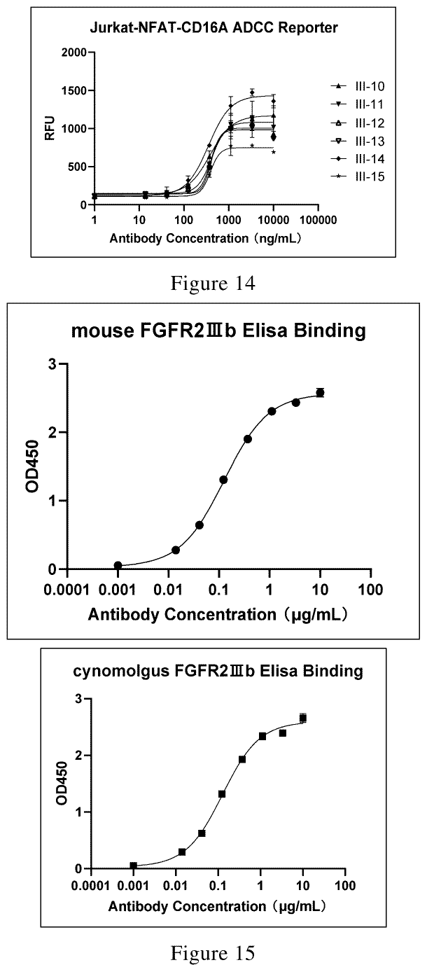

The inventors constructed a Jurkat-NFAT-Luc-CD16A stable cell line that expresses CD16 receptors and NFAT (Nuclear Factor of Activated T-cells) response elements. Using CHO-FGFR2IIIb as the target cell, the CHO-FGFR2IIIb cells were digested and centrifuged, and resuspended in culture medium at a density of 1.3E+06 cells/mL. After mixing, 60 μL of the cell suspension was transferred to a 384-well plate and cultured overnight. A gradient dilution solution of the antibody sample was prepared using complete culture medium. The supernatant was discarded from the 384-well plate, and 15 μL of the antibody solution was added to the plate and pre-incubated at 37° C. for 1 hour. Jurkat-NFAT-Luc-CD16A cells were resuspended in complete culture medium, 15 μL of cell suspension was added to each well, and the cells were placed in a 37° C. incubator for 4 hours. Prepare ONE-Glo™ Luciferase detection solution (Promega, Cat. No. E6110) and add it to a 384-well plate at 30 μL/well and allowed to react for 1-3 minutes. The plates were transferred to a multi-function microplate reader (Tecan Spark 20M) for analysis. As shown in FIG. 14, the EC₅₀ values of the antibodies were roughly equivalent. In terms of activation efficacy, all antibodies were able to activate immune cells, with III-14 demonstrating the strongest activation effect.

TABLE 10
ADCC effect of humanized antibodies
(RFU)

| Antibody | EC₅₀ (μg/mL) | Eₘₐₓ (RFU) |
|---|---|---|
| III-10 | 0.383 | 1170 |
| III-11 | 0.419 | 1082 |
| III-12 | 0.396 | 1007 |
| III-13 | 0.404 | 983 |
| III-14 | 0.352 | 1433 |
| III-15 | 0.388 | 749 |

### Example 13: Binding of Humanized Antibodies to Mouse/Cynomolgus Monkey FGFR2IIIb Protein

Coat a 96-well high-affinity plate with 100 μL/well of a 1 μg/mL mouse/cynomolgus monkey FGFR2 IIIb protein solution, and then incubate at 4° C. with shaking overnight. The next day, wash the plate three times with 300 μL of PBST (Tween 20:0.5% %) and then block with 100 μL/well of 5% BSA/PBS for 2 hours with shaking at room temperature. Wash the plate three times with 300 μL of PBST. Prepare a serial dilution of the antibody sample in PBS. Add 100 μL/well of the solution to the 96-well plate and shake at room temperature for 1 hour. Wash the plate three times with 300 μL of PBST. Prepare a secondary antibody solution (goat anti-human IgG HRP) and add 100 μL/well of the solution to the 96-well plate and shake at room temperature for 30 minutes. Wash the plate four times with 300 μL of PBST. Add 100 μL/well of TMB (tetramethylbenzidine) and develop color for 3 minutes. Add 100 μL/well 0.6N H₂SO₄ to stop color development and detect OD_{450 nm}.

The results are shown in FIG. 15. III-10 can bind to mouse and cynomolgus monkey FGFR2IIIb, and the EC₅₀ is shown in Table 11.

TABLE 11
Binding ability of humanized antibodies to mouse/
cynomolgus monkey FGFR2IIIb

| | | EC₅₀ (cynomolgus monkey |
|---|---|---|
| Antibody | EC₅₀ (mouse FGFR2b) | FGFR2b) |
| III-10 | 0.127 μg/mL | 0.128 μg/mL |

### Example 14: Detection of Antibody Affinity

Surface plasmon resonance (SPR) was used to detect antigen-antibody affinity. Using FGFR2 III b-his as the antigen, a certain concentration of antibody was incubated with a protein A sensor chip for antibody capture. In the antigen binding stage, gradiently diluted FGFR2IIIb-his protein was used as the mobile phase to bind to the antibody captured on the sensor chip. In the dissociation stage, HBS-EP buffer was used for continuous elution. The binding of the antibody to FGFR2IIIb-his on the sensor chip was quantitatively detected using Biacore 8k (GE Healthcare). The test results are shown in Table 12. The affinity of III-11, III-12, III-13, III-14, and III-15 to FGFR2IIIb-his were significantly improved compared to that of III-0 and III-10.

TABLE 12
Affinity of anti-human FGFR2IIIb antibodies to FGFR2IIIb-his

| Sample | ka (1/Ms) | kd (1/s) | KD (M) |
|---|---|---|---|
| III-0 | 1.06E+05 | 8.61E−03 | 8.13E−08 |
| III-10 | 1.39E+05 | 9.44E−03 | 6.81E−08 |
| III-11 | 1.82E+05 | 2.25E−04 | 1.24E−09 |
| III-12 | 1.58E+05 | 2.09E−04 | 1.32E−09 |
| III-13 | 1.41E+05 | 1.73E−04 | 1.23E−09 |
| III-14 | 1.06E+05 | 1.33E−04 | 1.26E−09 |
| III-15 | 1.49E+05 | 1.55E−04 | 1.04E−09 |

### Example 15: Efficacy Study of III-10 in the Mouse SNU-16 Tumor Model

For the SNU16 tumor model, SNU16 gastric cancer cells (5.0×10⁶) in serum-free medium were inoculated subcutaneously into the right upper abdomen of SCID female mice. When the tumor volume reached 170 mm³ (the fifth day after inoculation), the mice were randomly divided into groups (8 mice in each group), and intraperitoneally injected with the III-10 antibody and human albumin control twice weekly for 3 consecutive weeks at a concentration of 5 mg/kg. During the period, tumor volume, mouse body weight, and tumor volume were measured and recorded every 2-3 days.

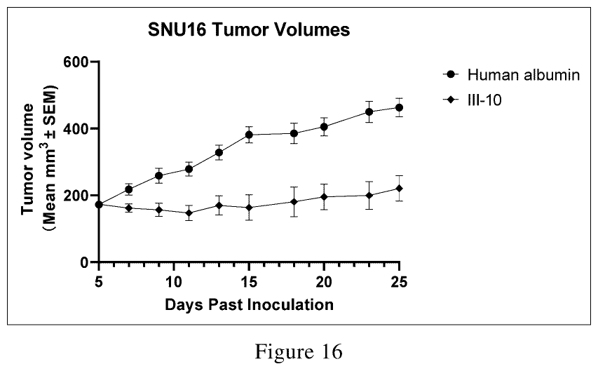

The results are shown in FIG. 16. III-10 exhibited significant ability to inhibit the growth of SNU16 tumors. Further experimental results showed that the antibodies of the present invention were also able to significantly inhibit SNU16 tumors even at low doses.

The sequences of the present invention are shown in Table 13 below.

TABLE 13
Sequence Listing

| SEQ ID | | |
|---|---|---|
| NO: | Sequence | Name |
| 1 | RAPYWTNTEKMEKRLHAVPAANTVKFRCPAGGNPMPT | FGFR2IIIb |
| | MRWLKNGKEFKQEHRIGGYKVRNQHWSLIMESVVPSD | (D2+D3) |
| | KGNYTCVVENEYGSINHTYHLDVVERSPHRPILQAGLP | |
| | ANASTVVGGDVEFVCKVYSDAQPHIQWIKHVEKNGSK | |
| | YGPDGLPYLKVLKHSGINSSNAEVLALFNVTEADAGEY | |
| | ICKVSNYIGQANQSAWLTVLPKQQAPGREKEITASPDYL | |
| | EHHHHHHHH | |
| 2 | RPSFSLVEDTTLEPEEPPTKYQISQPEVYVAAPGESLEVR | FGFR2 III b- |
| | CLLKDAAVISWTKDGVHLGPNNRTVLIGEYLQIKGATP | his |
| | RDSGLYACTASRTVDSETWYFMVNVTDAISSGDDEDD | (ECD) |
| | TDGAEDFVSENSNNKRAPYWTNTEKMEKRLHAVPAAN | |
| | TVKFRCPAGGNPMPTMRWLKNGKEFKQEHRIGGYKVR | |
| | NQHWSLIMESVVPSDKGNYTCVVENEYGSINHTYHLD | |
| | VVERSPHRPILQAGLPANASTVVGGDVEFVCKVYSDAQ | |
| | PHIQWIKHVEKNGSKYGPDGLPYLKVLKHSGINSSNAE | |
| | VLALFNVTEADAGEYICKVSNYIGQANQSAWLTVLPKQ | |
| | QAPGREKEITASPDYLEHHHHHHHH | |
| 3 | RPSFSLVEDTTLEPEEPPTKYQISQPEVYVAAPGESLEVR | FGFR2 III c- |
| | CLLKDAAVISWTKDGVHLGPNNRTVLIGEYLQIKGATP | his |
| | RDSGLYACTASRTVDSETWYFMVNVTDAISSGDDEDD | (ECD) |
| | TDGAEDFVSENSNNKRAPYWTNTEKMEKRLHAVPAAN | |
| | TVKFRCPAGGNPMPTMRWLKNGKEFKQEHRIGGYKVR | |
| | NQHWSLIMESVVPSDKGNYTCVVENEYGSINHTYHLD | |
| | VVERSPHRPILQAGLPANASTVVGGDVEFVCKVYSDAQ | |
| | PHIQWIKHVEKNGSKYGPDGLPYLKVLKAAGVNTTDK | |
| | EIEVLYIRNVTFEDAGEYTCLAGNSIGISFHSAWLTVLPA | |
| | PGREKEITASPDYLEHHHHHHHH | |
| 4 | QVQLQQPGAELVRPGASVKMSCKASGYTFANYNMHW | 27E9A11 |
| | VKQTPRQGLEWIGAIYPGNGDTSYNQKFKGKATLTAD | HCVR |
| | KSSSTAYMQLSSLTSEDSAVYYCARGDFGYWGQGTTL | |
| | TVSS | |
| 5 | DIVMTQSHKFMSTSVGDRVTITCKASQDVNIDVAWYQ | 27E9A11 |
| | QKPGQSPKLLIYSASYRYTGVPDRFTGSGSGTDFTFT | LCVR |
| | ISSVQAEDLAVYYCQQHYSTPYTFGGGTKLEIK | |
| 6 | NYNMH | 27E9A11 |
| | | VHCDR1 |
| | | (Kabat) |
| 7 | AIYPGNGDTSYNQKFKG | 27E9A11 |
| | | VHCDR2 |
| | | (Kabat) |
| 8 | GDFGY | 27E9A11 |
| | | VHCDR3 |
| | | (Kabat) |
| 9 | GYTFANYN | 27E9A11 |
| | | VHCDR1 |
| | | (IMGT) |
| 10 | IYPGNGDT | 27E9A11 |
| | | VHCDR2 |
| | | (IMGT) |
| 11 | ARGDFGY | 27E9A11 |
| | | VHCDR3 |
| | | (IMGT) |
| 12 | KASQDVNIDVA | VLCDR1 |
| | | (Kabat) |
| 13 | SASYRYT | VLCDR2 |
| | | (Kabat) |
| 14 | QQHYSTPYT | VLCDR3 |
| | | (Kabat/ |
| | | IMGT) |
| 15 | IYNMH | VHCDR1 |
| | | (Kabat) |
| 16 | AIYPDNGDTFYNQKFKG | VHCDR2 |
| | | (Kabat) |
| 17 | KASQDVNIDPA | VLCDR1 |
| | | (Kabat) |
| 18 | LASYRYT | VLCDR2 |
| | | (Kabat) |
| 19 | AIYPDNGDTSYNQKFKG | VHCDR2 |
| | | (Kabat) |
| 20 | QQTYSTPYT | VLCDR3 |
| | | (Kabat) |
| 21 | AIYPGNGDTFYNQKFKG | VHCDR2 |
| | | (Kabat) |
| 22 | QVQLQQPGAELVRPGASVKMSCKASGYTFANYNMHW | III-0 HC |
| | VKQTPRQGLEWIGAIYPGNGDTSYNQKFKGKATLTAD | |
| | KSSSTAYMQLSSLTSEDSAVYYCARGDFGYWGQGTTL | |
| | TVSSASTKGPSVFPLAPSSKSTSGGTAALGCLVKDYFPE | |
| | PVTVSWNSGALTSGVHTFPAVLQSSGLYSLSSVVTVPSS | |
| | SLGTQTYICNVNHKPSNTKVDKKVEPKSCDKTHTCPPC | |
| | PAPELLGGPSVFLFPPKPKDTLMISRTPEVTCVVVDVSH | |
| | EDPEVKFNWYVDGVEVHNAKTKPREEQYNSTYRVVSV | |
| | LTVLHQDWLNGKEYKCKVSNKALPAPIEKTISKAKGQP | |
| | REPQVYTLPPSRDELTKNQVSLTCLVKGFYPSDIAVEWE | |
| | SNGQPENNYKTTPPVLDSDGSFFLYSKLTVDKSRWQQG | |
| | NVFSCSVMHEALHNHYTQKSLSLSPGK | |
| 23 | DIVMTQSHKFMSTSVGDRVTITCKASQDVNIDVAWYQ | III-0 LC |
| | QKPGQSPKLLIYSASYRYTGVPDRFTGSGSGTDFTFTISS | |
| | VQAEDLAVYYCQQHYSTPYTFGGGTKLEIKRTVAAPSV | |
| | FIFPPSDEQLKSGTASVVCLLNNFYPREAKVQWKVDNA | |
| | LQSGNSQESVTEQDSKDSTYSLSSTLTLSKADYEKHKV | |
| | YACEVTHQGLSSPVTKSFNRGEC | |
| 24 | QVQLVQSGAEVKKPGSSVKVSCKASGYTFANYNMHW | III-10 |
| | VRQAPGQGLEWIGAIYPGNGDTSYNQKFKGKATLTAD | HCVR |
| | KSTSTAYMELSSLRSEDTAVYYCARGDFGYWGQGTLV | |
| | TVSS | |
| 25 | DIVMTQSPSSLSASVGDRVTITCKASQDVNIDVAWYQQ | III-10 |
| | KPGKAPKLLIYSASYRYTGVPSRFSGSGSGTDFTFTISSL | LCVR |
| | QPEDFATYYCQQHYSTPYTFGQGTKLEIK | |
| 26 | QVQLVQSGAEVKKPGSSVKVSCKASGYTFAIYNMHWV | III-11, III-15 |
| | RQAPGQGLEWIGAIYPDNGDTFYNQKFKGKATLTADKS | HCVR |
| | TSTAYMELSSLRSEDTAVYYCARGDFGYWGQGTLVTV | |
| | SS | |
| 27 | DIVMTQSPSSLSASVGDRVTITCKASQDVNIDPAWYQQ | III-11 |
| | KPGKAPKLLIYLASYRYTGVPSRFSGSGSGTDFTFTISSL | LCVR |
| | QPEDFATYYCQQHYSTPYTFGQGTKLEIK | |
| 28 | QVQLVQSGAEVKKPGSSVKVSCKASGYTFANYNMHW | III-12 |
| | VRQAPGQGLEWIGAIYPDNGDTSYNQKFKGKATLTAD | HCVR |
| | KSTSTAYMELSSLRSEDTAVYYCARGDFGYWGQGTLV | |
| | TVSS | |
| 29 | DIVMTQSPSSLSASVGDRVTITCKASQDVNIDVAWYQQ | III-12, III- |
| | KPGKAPKLLIYLASYRYTGVPSRFSGSGSGTDFTFTISSL | 13, III-14, |
| | QPEDFATYYCQQTYSTPYTFGQGTKLEIK | III-15 |
| | | LCVR |
| 30 | QVQLVQSGAEVKKPGSSVKVSCKASGYTFAIYNMHWV | III-13 |
| | RQAPGQGLEWIGAIYPDNGDTSYNQKFKGKATLTADKS | HCVR |
| | TSTAYMELSSLRSEDTAVYYCARGDFGYWGQGTLVTV | |
| | SS | |
| 31 | QVQLVQSGAEVKKPGSSVKVSCKASGYTFAIYNMHWV | III-14 |
| | RQAPGQGLEWIGAIYPGNGDTFYNQKFKGKATLTADKS | HCVR |
| | TSTAYMELSSLRSEDTAVYYCARGDFGYWGQGTLVTV | |
| | SS | |
| 32 | QVQLVQSGAEVKKPGSSVKVSCKASGYTFANYNMHW | III-10 HC |
| | VRQAPGQGLEWIGAIYPGNGDTSYNQKFKGKATLTAD | |
| | KSTSTAYMELSSLRSEDTAVYYCARGDFGYWGQGTLV | |
| | TVSSASTKGPSVFPLAPSSKSTSGGTAALGCLVKDYFPE | |
| | PVTVSWNSGALTSGVHTFPAVLQSSGLYSLSSVVTVPSS | |
| | SLGTQTYICNVNHKPSNTKVDKKVEPKSCDKTHTCPPC | |
| | PAPELLGGPSVFLFPPKPKDTLMISRTPEVTCVVVDVSH | |
| | EDPEVKFNWYVDGVEVHNAKTKPREEQYNSTYRVVSV | |
| | LTVLHQDWLNGKEYKCKVSNKALPAPIEKTISKAKGQP | |
| | REPQVYTLPPSRDELTKNQVSLTCLVKGFYPSDIAVEWE | |
| | SNGQPENNYKTTPPVLDSDGSFFLYSKLTVDKSRWQQG | |
| | NVFSCSVMHEALHNHYTQKSLSLSPGK | |
| 33 | DIVMTQSPSSLSASVGDRVTITCKASQDVNIDVAWYQQ | III-10 LC |
| | KPGKAPKLLIYSASYRYTGVPSRFSGSGSGTDFTFTISSL | |
| | QPEDFATYYCQQHYSTPYTFGQGTKLEIKRTVAAPSVFI | |
| | FPPSDEQLKSGTASVVCLLNNFYPREAKVQWKVDNAL | |
| | QSGNSQESVTEQDSKDSTYSLSSTLTLSKADYEKHKVY | |
| | ACEVTHQGLSSPVTKSFNRGEC | |
| 34 | QVQLVQSGAEVKKPGSSVKVSCKASGYTFAIYNMHWV | III-11, |
| | RQAPGQGLEWIGAIYPDNGDTFYNQKFKGKATLTADKS | III-15 HC |
| | TSTAYMELSSLRSEDTAVYYCARGDFGYWGQGTLVTV | |
| | SSASTKGPSVFPLAPSSKSTSGGTAALGCLVKDYFPEPV | |
| | TVSWNSGALTSGVHTFPAVLQSSGLYSLSSVVTVPSSSL | |
| | GTQTYICNVNHKPSNTKVDKKVEPKSCDKTHTCPPCPA | |
| | PELLGGPSVFLFPPKPKDTLMISRTPEVTCVVVDVSHED | |
| | PEVKFNWYVDGVEVHNAKTKPREEQYNSTYRVVSVLT | |
| | VLHQDWLNGKEYKCKVSNKALPAPIEKTISKAKGQPRE | |
| | PQVYTLPPSREEMTKNQVSLTCLVKGFYPSDIAVEWES | |
| | NGQPENNYKTTPPVLDSDGSFFLYSKLTVDKSRWQQGN | |
| | VFSCSVMHEALHNHYTQKSLSLSPGK | |
| 35 | DIVMTQSPSSLSASVGDRVTITCKASQDVNIDPAWYQQ | III-11 LC |
| | KPGKAPKLLIYLASYRYTGVPSRFSGSGSGTDFTFTISSL | |
| | QPEDFATYYCQQHYSTPYTFGQGTKLEIKRTVAAPSVFI | |
| | FPPSDEQLKSGTASVVCLLNNFYPREAKVQWKVDNAL | |
| | QSGNSQESVTEQDSKDSTYSLSSTLTLSKADYEKHKVY | |
| | ACEVTHQGLSSPVTKSFNRGEC | |
| 36 | QVQLVQSGAEVKKPGSSVKVSCKASGYTFANYNMHW | III-12 HC |
| | VRQAPGQGLEWIGAIYPDNGDTSYNQKFKGKATLTAD | |
| | KSTSTAYMELSSLRSEDTAVYYCARGDFGYWGQGTLV | |
| | TVSSASTKGPSVFPLAPSSKSTSGGTAALGCLVKDYFPE | |
| | PVTVSWNSGALTSGVHTFPAVLQSSGLYSLSSVVTVPSS | |
| | SLGTQTYICNVNHKPSNTKVDKKVEPKSCDKTHTCPPC | |
| | PAPELLGGPSVFLFPPKPKDTLMISRTPEVTCVVVDVSH | |
| | EDPEVKFNWYVDGVEVHNAKTKPREEQYNSTYRVVSV | |
| | LTVLHQDWLNGKEYKCKVSNKALPAPIEKTISKAKGQP | |
| | REPQVYTLPPSREEMTKNQVSLTCLVKGFYPSDIAVEW | |
| | ESNGQPENNYKTTPPVLDSDGSFFLYSKLTVDKSRWQQ | |
| | GNVFSCSVMHEALHNHYTQKSLSLSPGK | |
| 37 | DIVMTQSPSSLSASVGDRVTITCKASQDVNIDVAWYQQ | III-12, III- |
| | KPGKAPKLLIYLASYRYTGVPSRFSGSGSGTDFTFTISSL | 13, III-14, |
| | QPEDFATYYCQQTYSTPYTFGQGTKLEIKRTVAAPSVFI | III-15 LC |
| | FPPSDEQLKSGTASVVCLLNNFYPREAKVQWKVDNAL | |
| | QSGNSQESVTEQDSKDSTYSLSSTLTLSKADYEKHKVY | |
| | ACEVTHQGLSSPVTKSFNRGEC | |
| 38 | QVQLVQSGAEVKKPGSSVKVSCKASGYTFAIYNMHWV | III-13 HC |
| | RQAPGQGLEWIGAIYPDNGDTSYNQKFKGKATLTADKS | |
| | TSTAYMELSSLRSEDTAVYYCARGDFGYWGQGTLVTV | |
| | SSASTKGPSVFPLAPSSKSTSGGTAALGCLVKDYFPEPV | |
| | TVSWNSGALTSGVHTFPAVLQSSGLYSLSSVVTVPSSSL | |
| | GTQTYICNVNHKPSNTKVDKKVEPKSCDKTHTCPPCPA | |
| | PELLGGPSVFLFPPKPKDTLMISRTPEVTCVVVDVSHED | |
| | PEVKFNWYVDGVEVHNAKTKPREEQYNSTYRVVSVLT | |
| | VLHQDWLNGKEYKCKVSNKALPAPIEKTISKAKGQPRE | |
| | PQVYTLPPSREEMTKNQVSLTCLVKGFYPSDIAVEWES | |
| | NGQPENNYKTTPPVLDSDGSFFLYSKLTVDKSRWQQGN | |
| | VFSCSVMHEALHNHYTQKSLSLSPGK | |
| 39 | QVQLVQSGAEVKKPGSSVKVSCKASGYTFAIYNMHWV | III-14 HC |
| | RQAPGQGLEWIGAIYPGNGDTFYNQKFKGKATLTADKS | |
| | TSTAYMELSSLRSEDTAVYYCARGDFGYWGQGTLVTV | |
| | SSASTKGPSVFPLAPSSKSTSGGTAALGCLVKDYFPEPV | |
| | TVSWNSGALTSGVHTFPAVLQSSGLYSLSSVVTVPSSSL | |
| | GTQTYICNVNHKPSNTKVDKKVEPKSCDKTHTCPPCPA | |
| | PELLGGPSVFLFPPKPKDTLMISRTPEVTCVVVDVSHED | |
| | PEVKFNWYVDGVEVHNAKTKPREEQYNSTYRVVSVLT | |
| | VLHQDWLNGKEYKCKVSNKALPAPIEKTISKAKGQPRE | |
| | PQVYTLPPSREEMTKNQVSLTCLVKGFYPSDIAVEWES | |
| | NGQPENNYKTTPPVLDSDGSFFLYSKLTVDKSRWQQGN | |
| | VFSCSVMHEALHNHYTQKSLSLSPGK | |
| 40 | QDVNID | 27E9A11 |
| | | VLCDR1 |
| | | (IMGT) |

All literatures mentioned in the present application are incorporated herein by reference, as though each one is individually incorporated by reference. In addition, it should also be understood that, after reading the above teachings of the present invention, those skilled in the art can make various changes or modifications, equivalents of which falls in the scope of claims as defined in the appended claims.

1. An anti-FGFR2IIIb antibody comprising a heavy chain and a light chain, wherein, the variable region of the heavy chain has complementarity determining regions (CDR) selected from the group consisting of:
(1) VH-CDR1 shown in SEQ ID NO: 6, VH-CDR2 shown in SEQ ID NO: 7, and VH-CDR3 shown in SEQ ID NO: 8, wherein the CDRs are defined according to the Kabat rule;
(2) VH-CDR1 shown in SEQ ID NO: 15, VH-CDR2 shown in SEQ ID NO: 16, and VH-CDR3 shown in SEQ ID NO: 8, wherein the CDRs are defined according to the Kabat rule;
(3) VH-CDR1 shown in SEQ ID NO: 6, VH-CDR2 shown in SEQ ID NO: 19, and VH-CDR3 shown in SEQ ID NO: 8, wherein the CDRs are defined according to the Kabat rule;
(4) VH-CDR1 shown in SEQ ID NO: 15, VH-CDR2 shown in SEQ ID NO: 19, and VH-CDR3 shown in SEQ ID NO: 8, wherein the CDRs are defined according to the Kabat rule;
(5) VH-CDR1 shown in SEQ ID NO: 15, VH-CDR2 shown in SEQ ID NO: 21, and VH-CDR3 shown in SEQ ID NO: 8, wherein the CDRs are defined according to the Kabat rule; and
(6) VH-CDR1 shown in SEQ ID NO: 9, VH-CDR2 shown in SEQ ID NO: 10, and VH-CDR3 shown in SEQ ID NO: 11, wherein the CDRs are defined according to the IMGT rule;
Furthermore, the variable region of the light chain has complementarity determining regions (CDR) selected from the group consisting of:
(1) VL-CDR1 shown in SEQ ID NO: 12, VL-CDR2 shown in SEQ ID NO: 13, and VL-CDR3 shown in SEQ ID NO: 14, wherein the CDRs are defined according to the Kabat rule;
(2) VL-CDR1 shown in SEQ ID NO: 17, VL-CDR2 shown in SEQ ID NO: 18, and VL-CDR3 shown in SEQ ID NO: 14, wherein the CDRs are defined according to the Kabat rule;
(3) VL-CDR1 shown in SEQ ID NO: 12, VL-CDR2 shown in SEQ ID NO: 18, and VL-CDR3 shown in SEQ ID NO: 20, wherein the CDRs are defined according to the Kabat rule; and
(4) VL-CDR1 shown in SEQ ID NO:40, VL-CDR2 represented by SAS, and VL-CDR3 shown in SEQ ID NO:14, wherein the CDRs are defined according to IMGT rule;
Furthermore, any one of the amino acid sequences in the above-mentioned CDR sequences also includes a derivative sequence that optionally has 1-2 amino acids added, deleted, modified, and/or substituted, and enables the derivative antibody composed of the heavy chain and light chain containing the derived CDR sequence to retain the binding affinity for FGFR2IIIb or its derivative protein.

2. The antibody of claim 1, wherein, the antibody has heavy chain variable regions CDRs (VH-CDR) and light chain variable regions CDRs (VL-CDR) selected from the group consisting of:
(1) VH-CDR1 shown in SEQ ID NO: 6, VH-CDR2 shown in SEQ ID NO: 7, and VH-CDR3 shown in SEQ ID NO: 8, VL-CDR1 shown in SEQ ID NO: 12, VL-CDR2 shown in SEQ ID NO: 13, and VL-CDR3 shown in SEQ ID NO: 14, wherein the CDRs are defined according to the Kabat rule;
(2) VH-CDR1 shown in SEQ ID NO: 15, VH-CDR2 shown in SEQ ID NO: 16, and VH-CDR3 shown in SEQ ID NO: 8, VL-CDR1 shown in SEQ ID NO: 17, VL-CDR2 shown in SEQ ID NO: 18, and VL-CDR3 shown in SEQ ID NO: 14, wherein the CDRs are defined according to the Kabat rule;
(3) VH-CDR1 shown in SEQ ID NO: 6, VH-CDR2 shown in SEQ ID NO: 19, and VH-CDR3 shown in SEQ ID NO: 8, VL-CDR1 shown in SEQ ID NO: 12, VL-CDR2 shown in SEQ ID NO: 18, and VL-CDR3 shown in SEQ ID NO: 20, wherein the CDRs are defined according to the Kabat rule;
(4) VH-CDR1 shown in SEQ ID NO: 15, VH-CDR2 shown in SEQ ID NO: 19, and VH-CDR3 shown in SEQ ID NO: 8, VL-CDR1 shown in SEQ ID NO: 12, VL-CDR2 shown in SEQ ID NO: 18, and VL-CDR3 shown in SEQ ID NO: 20, wherein the CDRs are defined according to the Kabat rule;
(5) VH-CDR1 shown in SEQ ID NO: 15, VH-CDR2 shown in SEQ ID NO: 21, and VH-CDR3 shown in SEQ ID NO: 8, VL-CDR1 shown in SEQ ID NO: 12, VL-CDR2 shown in SEQ ID NO: 18, and VL-CDR3 shown in SEQ ID NO: 20, wherein the CDRs are defined according to the Kabat rule;
(6) VH-CDR1 shown in SEQ ID NO: 15, VH-CDR2 shown in SEQ ID NO: 16, and VH-CDR3 shown in SEQ ID NO: 8, VL-CDR1 shown in SEQ ID NO: 12, VL-CDR2 shown in SEQ ID NO: 18, and VL-CDR3 shown in SEQ ID NO: 20, wherein the CDRs are defined according to the Kabat rule; and
(7) VH-CDR1 shown in SEQ ID NO: 9, VH-CDR2 shown in SEQ ID NO: 10, and VH-CDR3 shown in SEQ ID NO: 11, VL-CDR1 shown in SEQ ID NO:40, VL-CDR2 represented by SAS, and VL-CDR3 shown in SEQ ID NO:14, wherein the CDRs are defined according to the IMGT rule.

3. The antibody of claim 1, wherein, the heavy chain variable region of the antibody comprises the amino acid sequence of SEQ ID NO: 24, 26, 28, 30, 31 or 4 or the amino acid sequence having at least 80%, 85%, 90%, 91%, 92%, 93%, 94%, 95%, 96%, 97%, 98% or 99% sequence homology or sequence identity thereto; and/or the light chain variable region of the antibody comprises the amino acid sequence of SEQ ID NO: 25, 27, 29 or 5 or the amino acid sequence having at least 80%, 85%, 90%, 91%, 92%, 93%, 94%, 95%, 96%, 97%, 98% or 99% sequence homology or sequence identity thereto.

4. The antibody of claim 1, wherein, the antibody has:
(1) the heavy chain variable region shown in SEQ ID NO: 24 and the light chain variable region shown in SEQ ID NO: 25;
(2) the heavy chain variable region shown in SEQ ID NO: 26 and the light chain variable region shown in SEQ ID NO: 27;
(3) the heavy chain variable region shown in SEQ ID NO: 28 and the light chain variable region shown in SEQ ID NO: 29;
(4) the heavy chain variable region shown in SEQ ID NO:30 and the light chain variable region shown in SEQ ID NO:29;
(5) the heavy chain variable region shown in SEQ ID NO:31 and the light chain variable region shown in SEQ ID NO:29;
(6) the heavy chain variable region shown in SEQ ID NO: 26 and the light chain variable region shown in SEQ ID NO: 29; or
(7) The heavy chain variable region shown in SEQ ID NO: 4 and the light chain variable region shown in SEQ ID NO: 5.

5. The antibody of claim 1, wherein, the amino acid sequence of the heavy chain is shown in SEQ ID NO: 32, 34, 36, 38, 39 or 22 or has at least 80%, 85%, 90%, 91%, 92%, 93%, 94%, 95%, 96%, 97%, 98% or 99% sequence homology or sequence identity thereto; and/or the amino acid sequence of the light chain is shown in SEQ ID NO: 33, 35, 37 or 23 or has at least 80%, 85%, 90%, 91%, 92%, 93%, 94%, 95%, 96%, 97%, 98% or 99% sequence homology or sequence identity thereto.

6. The antibody of claim 1, wherein, the antibody has:
(1) a heavy chain shown in SEQ ID NO: 32 and a light chain shown in SEQ ID NO: 33;
(2) a heavy chain shown in SEQ ID NO:34 and a light chain shown in SEQ ID NO:35;
(3) a heavy chain shown in SEQ ID NO: 36 and a light chain shown in SEQ ID NO: 37;
(4) a heavy chain shown in SEQ ID NO: 38 and a light chain shown in SEQ ID NO: 37;
(5) a heavy chain shown in SEQ ID NO: 39 and a light chain shown in SEQ ID NO: 37;
(6) a heavy chain shown in SEQ ID NO: 34 and a light chain shown in SEQ ID NO: 37; or
(7) a heavy chain shown in SEQ ID NO: 22 and a light chain shown in SEQ ID NO: 23.

7. A recombinant protein, wherein the recombinant protein comprises:
(i) the antibody of claim 1; and
(ii) optional tag sequence for assisting expression and/or purification.

8. A chimeric antigen receptor (CAR) construct, wherein the antigen binding region of the chimeric antigen receptor construct comprises a single-chain variable fragment (scFv) that specifically binds to FGFR2IIIb or its derivative protein, and the scFv has the heavy chain variable region and light chain variable region of the antibody of claim 1.

9. A recombinant immune cell, which expresses an exogenous CAR construct of claim 8.

10. A pharmaceutical composition comprising:
(i) the antibody of claim 1 as an active ingredient; and
(ii) a pharmaceutically acceptable carrier.

11-12. (canceled)

13. A method for preventing and/or treating a disease associated with abnormal expression or function of FGFR2IIIb or its derivative protein, which comprises administering an effective amount of the antibody of claim 1 to a subject in need thereof.

14. The method according to claim 13, wherein, the disease is cancer.

15. The method of claim 14, wherein, the cancer is selected from gastric cancer, esophageal cancer, colorectal cancer, breast cancer, ovarian cancer, endometrial cancer, endometrioid adenocarcinoma, cholangiocarcinoma, lung cancer, and non-small cell lung cancer.

16. A polynucleotide encoding
the antibody of claim 1.

17. A vector comprising the polynucleotide of claim 16.

18. An engineered host cell, wherein, the host cell comprises the vector of claim 17.

19. A detection plate, wherein, the detection plate includes a substrate and a test strip, and the test strip contains the antibody of claim 1.

20. A method for detecting FGFR2IIIb or its derivative protein in a sample, wherein the method comprises the steps:
(1) contacting the sample with the antibody of claim 1; and
(2) detecting the formation of an antigen-antibody complex, wherein the formation of the complex indicates the presence of FGFR2IIIb or its derivative protein in the sample.


SEQUENCE LISTING (40 sequences)

SEQ ID NO: 1 - protein, 235 aa, synthetic construct
  source 1..235: note = FGFR2IIIb D2+D3
RAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPDYLEHHHHHHHH

SEQ ID NO: 2 - protein, 365 aa, synthetic construct
  source 1..365: note = FGFR2IIIb-his
RPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPDYLEHHHHHHHH

SEQ ID NO: 3 - protein, 364 aa, synthetic construct
  source 1..364: note = FGFR2IIIc-his
RPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEHHHHHHHH

SEQ ID NO: 4 - protein, 114 aa, Mus musculus
QVQLQQPGAELVRPGASVKMSCKASGYTFANYNMHWVKQTPRQGLEWIGAIYPGNGDTSYNQKFKGKATLTADKSSSTAYMQLSSLTSEDSAVYYCARGDFGYWGQGTTLTVSS

SEQ ID NO: 5 - protein, 107 aa, Mus musculus
DIVMTQSHKFMSTSVGDRVTITCKASQDVNIDVAWYQQKPGQSPKLLIYSASYRYTGVPDRFTGSGSGTDFTFTISSVQAEDLAVYYCQQHYSTPYTFGGGTKLEIK

SEQ ID NO: 6 - protein, 5 aa, Mus musculus
NYNMH

SEQ ID NO: 7 - protein, 17 aa, Mus musculus
AIYPGNGDTSYNQKFKG

SEQ ID NO: 8 - protein, 5 aa, Mus musculus
GDFGY

SEQ ID NO: 9 - protein, 8 aa, Mus musculus
GYTFANYN

SEQ ID NO: 10 - protein, 8 aa, Mus musculus
IYPGNGDT

SEQ ID NO: 11 - protein, 7 aa, Mus musculus
ARGDFGY

SEQ ID NO: 12 - protein, 11 aa, synthetic construct
  source 1..11: note = VLCDR1
KASQDVNIDVA

SEQ ID NO: 13 - protein, 7 aa, synthetic construct
  source 1..7: note = VLCDR2
SASYRYT

SEQ ID NO: 14 - protein, 9 aa, synthetic construct
  source 1..9: note = VLCDR3
QQHYSTPYT

SEQ ID NO: 15 - protein, 5 aa, synthetic construct
  source 1..5: note = VHCDR1
IYNMH

SEQ ID NO: 16 - protein, 17 aa, synthetic construct
  source 1..17: note = VHCDR2
AIYPDNGDTFYNQKFKG

SEQ ID NO: 17 - protein, 11 aa, synthetic construct
  source 1..11: note = VLCDR1
KASQDVNIDPA

SEQ ID NO: 18 - protein, 7 aa, synthetic construct
  source 1..7: note = VLCDR2
LASYRYT

SEQ ID NO: 19 - protein, 17 aa, synthetic construct
  source 1..17: note = VHCDR2
AIYPDNGDTSYNQKFKG

SEQ ID NO: 20 - protein, 9 aa, synthetic construct
  source 1..9: note = VLCDR3
QQTYSTPYT

SEQ ID NO: 21 - protein, 17 aa, synthetic construct
  source 1..17: note = VHCDR2
AIYPGNGDTFYNQKFKG

SEQ ID NO: 22 - protein, 444 aa, synthetic construct
  source 1..444: note = III-0 HC
QVQLQQPGAELVRPGASVKMSCKASGYTFANYNMHWVKQTPRQGLEWIGAIYPGNGDTSYNQKFKGKATLTADKSSSTAYMQLSSLTSEDSAVYYCARGDFGYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 214 aa, synthetic construct
  source 1..214: note = III-0 LC
DIVMTQSHKFMSTSVGDRVTITCKASQDVNIDVAWYQQKPGQSPKLLIYSASYRYTGVPDRFTGSGSGTDFTFTISSVQAEDLAVYYCQQHYSTPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 114 aa, synthetic construct
  source 1..114: note = III-10 HCVR
QVQLVQSGAEVKKPGSSVKVSCKASGYTFANYNMHWVRQAPGQGLEWIGAIYPGNGDTSYNQKFKGKATLTADKSTSTAYMELSSLRSEDTAVYYCARGDFGYWGQGTLVTVSS

SEQ ID NO: 25 - protein, 107 aa, synthetic construct
  source 1..107: note = III-10 LCVR
DIVMTQSPSSLSASVGDRVTITCKASQDVNIDVAWYQQKPGKAPKLLIYSASYRYTGVPSRFSGSGSGTDFTFTISSLQPEDFATYYCQQHYSTPYTFGQGTKLEIK

SEQ ID NO: 26 - protein, 114 aa, synthetic construct
  source 1..114: note = III-11, III-15 HCVR
QVQLVQSGAEVKKPGSSVKVSCKASGYTFAIYNMHWVRQAPGQGLEWIGAIYPDNGDTFYNQKFKGKATLTADKSTSTAYMELSSLRSEDTAVYYCARGDFGYWGQGTLVTVSS

SEQ ID NO: 27 - protein, 107 aa, synthetic construct
  source 1..107: note = III-11 LCVR
DIVMTQSPSSLSASVGDRVTITCKASQDVNIDPAWYQQKPGKAPKLLIYLASYRYTGVPSRFSGSGSGTDFTFTISSLQPEDFATYYCQQHYSTPYTFGQGTKLEIK

SEQ ID NO: 28 - protein, 114 aa, synthetic construct
  source 1..114: note = III-12 HCVR
QVQLVQSGAEVKKPGSSVKVSCKASGYTFANYNMHWVRQAPGQGLEWIGAIYPDNGDTSYNQKFKGKATLTADKSTSTAYMELSSLRSEDTAVYYCARGDFGYWGQGTLVTVSS

SEQ ID NO: 29 - protein, 107 aa, synthetic construct
  source 1..107: note = III-12, III-13, III-14, III-15 LCVR
DIVMTQSPSSLSASVGDRVTITCKASQDVNIDVAWYQQKPGKAPKLLIYLASYRYTGVPSRFSGSGSGTDFTFTISSLQPEDFATYYCQQTYSTPYTFGQGTKLEIK

SEQ ID NO: 30 - protein, 114 aa, synthetic construct
  source 1..114: note = III-13 HCVR
QVQLVQSGAEVKKPGSSVKVSCKASGYTFAIYNMHWVRQAPGQGLEWIGAIYPDNGDTSYNQKFKGKATLTADKSTSTAYMELSSLRSEDTAVYYCARGDFGYWGQGTLVTVSS

SEQ ID NO: 31 - protein, 114 aa, synthetic construct
  source 1..114: note = III-14 HCVR
QVQLVQSGAEVKKPGSSVKVSCKASGYTFAIYNMHWVRQAPGQGLEWIGAIYPGNGDTFYNQKFKGKATLTADKSTSTAYMELSSLRSEDTAVYYCARGDFGYWGQGTLVTVSS

SEQ ID NO: 32 - protein, 444 aa, synthetic construct
  source 1..444: note = III-10 HC
QVQLVQSGAEVKKPGSSVKVSCKASGYTFANYNMHWVRQAPGQGLEWIGAIYPGNGDTSYNQKFKGKATLTADKSTSTAYMELSSLRSEDTAVYYCARGDFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 214 aa, synthetic construct
  source 1..214: note = III-10 LC
DIVMTQSPSSLSASVGDRVTITCKASQDVNIDVAWYQQKPGKAPKLLIYSASYRYTGVPSRFSGSGSGTDFTFTISSLQPEDFATYYCQQHYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 444 aa, synthetic construct
  source 1..444: note = III-11,III-15 HC
QVQLVQSGAEVKKPGSSVKVSCKASGYTFAIYNMHWVRQAPGQGLEWIGAIYPDNGDTFYNQKFKGKATLTADKSTSTAYMELSSLRSEDTAVYYCARGDFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 214 aa, synthetic construct
  source 1..214: note = III-11 LC
DIVMTQSPSSLSASVGDRVTITCKASQDVNIDPAWYQQKPGKAPKLLIYLASYRYTGVPSRFSGSGSGTDFTFTISSLQPEDFATYYCQQHYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 444 aa, synthetic construct
  source 1..444: note = III-12 HC
QVQLVQSGAEVKKPGSSVKVSCKASGYTFANYNMHWVRQAPGQGLEWIGAIYPDNGDTSYNQKFKGKATLTADKSTSTAYMELSSLRSEDTAVYYCARGDFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 214 aa, synthetic construct
  source 1..214: note = III-12, III-13, III-14, III-15 LC
DIVMTQSPSSLSASVGDRVTITCKASQDVNIDVAWYQQKPGKAPKLLIYLASYRYTGVPSRFSGSGSGTDFTFTISSLQPEDFATYYCQQTYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 444 aa, synthetic construct
  source 1..444: note = III-13 HC
QVQLVQSGAEVKKPGSSVKVSCKASGYTFAIYNMHWVRQAPGQGLEWIGAIYPDNGDTSYNQKFKGKATLTADKSTSTAYMELSSLRSEDTAVYYCARGDFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 444 aa, synthetic construct
  source 1..444: note = III-14 HC
QVQLVQSGAEVKKPGSSVKVSCKASGYTFAIYNMHWVRQAPGQGLEWIGAIYPGNGDTFYNQKFKGKATLTADKSTSTAYMELSSLRSEDTAVYYCARGDFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 6 aa, Mus musculus
QDVNID